(19) United States
(12) Patent Application Publication
(10) Pub. No.: US 2019/0282640 A1
(43) Pub. Date: Sep. 19, 2019

(54) PSEUDOTYPED ONCOLYTIC RHABDOVIRUSES AND THEIR USE IN COMBINATION THERAPY

(71) Applicant: TURNSTONE LIMITED PARTNERSHIP, Toronto, Ontario (CA)

(72) Inventors: Laura Evgin, Ottawa, Ontario (CA); Lawton Stubbert, Ottawa, Ontario (CA); Michael Huh, Gatineau, Quebec (CA); John Bell, Ottawa, Ontario (CA)

(21) Appl. No.: 16/303,077

(22) PCT Filed: May 18, 2017
(86) PCT No.: PCT/CA2017/050605

§ 371 Date: Nov. 19, 2018

Related U.S. Application Data
(60) Provisional application No. 62/338,940, filed on May 19, 2016.

(51) Int. Cl. A61K 35/766; C12N 7/00; A61K 9/00; C12N 15/86; C07K 14/145

(52) U.S. Cl. CPC A61K 35/766; C12N 7/00; A61K 45/06; C12N 15/86; C07K 14/145; A61K 9/0019

(57) ABSTRACT

Embodiments of the invention include compositions and methods related to replicative oncolytic rhabdoviruses pseudotyped with an arenavirus glycoprotein and their use as anti-cancer therapeutics particularly in combination with complement inhibitors.

### I. CROSS-REFERENCE TO RELATED APPLICATIONS

This application claims the benefit of U.S. Provisional Patent Application Ser. No. 62/338,940, filed May 19, 2016, the full disclosure of which is incorporated herein by reference.

### II. INCORPORATION BY REFERENCE OF A SEQUENCE LISTING PROVIDED AS A TEXT FILE

A Sequence Listing is provided herewith as a text file, “PAT 104044W-90 Sequence Listing.txt” created on May 15, 2017 and having a size of 12.6 KB. The contents of the text file are incorporated by reference herein in its entirety.

### III. FIELD OF THE INVENTION

This invention relates generally to virology and medicine. In certain aspects the invention relates to oncolytic viruses, particularly chimeric oncolytic rhabdoviruses and their use in combination with complement inhibitors for treating cancer.

### IV. BACKGROUND

Oncolytic viruses specifically infect, replicate in, and kill malignant cells leaving normal tissues unaffected. Several oncolytic viruses have reached advanced stages of clinical evaluation for the treatment of a variety of neoplasms.

Rhabdoviruses, including vesicular stomatitis virus (VSV) and Maraba virus (MRB), are two examples of oncolytic rhbadoviruses that have been studied extensively pre-clinically. Rhabodviruses are promising clinical candidates as the viruses show no genetic reassortment, integration into host genome or malignant transformation potential. The viruses are lytic across a broad range of tumor cells and are highly sensitive to Type 1 interferon, making the therapeutic index quite large. Additionally, human infections are rare and usually asymptomatic and there is virtually no pre-existing immunity in humans. Oncolytic VSV and MRB are currently being evaluated clinically in Phase I human clinical trials.

Several studies have used mouse models to demonstrate that, after the initial in vivo treatment with oncolytic rhabdoviruses, strong neutralizing antibody responses develop that drastically reduce the efficacy of subsequent doses of virus. A significant advance in the art would result from treatment methods which overcome antibody neutralization of circulating oncolytic rhabdovirus.

### SUMMARY OF THE INVENTION

In several embodiments, a pseudotyped replicative oncolytic rhabdovirus is provided comprising an arenavirus envelope glycoprotein in place of the rhabodvirus glycoprotein as well as a pharmaceutical composition comprising a pseudotyped replicative oncolytic rhabdovirus comprising an arenavirus glycoprotein and a pharmaceutically acceptable carrier. In some embodiments, the pseudotyped replicative oncolytic rhabdovirus is a wild type or recombinant vesiculovirus, particularly a wild type or recombinant vesicular stomatitis virus (VSV) or Maraba virus (MRB) with an arenavirus glycoprotein replacing the VSV or MRB glycoprotein. In some embodiments, the pseudotyped oncolytic rhabdovirus is a VSV or MRB comprising one or more genetic modifications that increase tumor selectivity and/or oncolytic effect of the virus. In other preferred embodiments, the arenavirus glycoprotein is a lymphocytic choriomeningtitis virus (LCMV) glycoprotein, a Lassa virus glycoprotein, a Junin virus glycoprotein or a variant thereof. In particularly preferred embodiments, a pseudotyped oncolytic VSV or Maraba virus with a Lassa or Junin glycoprotein replacing the VSV or Maraba glycoprotein is provided. In some embodiments, the pseudotyped replicative oncolytic rhabdovirus exhibits reduced neurotropism compared to a non-pseudotyped replicative oncolytic rhabodvirus with the same genetic background. In other embodiments, the pseudotyped replicative oncolytic rhabdovirus comprises heterologous nucleic acid sequence encoding one or more tumor antigens such as those mentioned in paragraphs [0071]-[0082] of WIPO publication no. WO 2014/127478 and paragraph [0042] of U.S. Patent Application Publication No. 2012/0014990, the contents of both of which are incorporated herein by reference and/or comprises heterologous nucleic acid sequence encoding one or more cytokines and/or comprises heterologous nucleic acid sequence encoding one or more immune checkpoint inhibitors.

In other embodiments, a method for treating and/or preventing cancer and/or treating and/or preventing a metastasis is provided comprising administering to a mammal in need thereof an effective amount of a pseudotyped replicative oncolytic rhabdovirus comprising an arenavirus glycoprotein. In preferred embodiments, the oncolytic rhabdovirus is a VSV or Maraba virus pseudotyped with a Lassa virus or Junin virus glycoprotein and the mammal is a human. Preferably, the mammal is administered multiple doses (2, 3, 4, 5, 6 or more doses) of the pseudotyped replicative oncolytic rhabodvirus by a systemic (e.g. intravascular) and/or intratumoral route of administration. In other preferred embodiments, the cancer to be treated and/or prevented is selected from liver cancer, brain cancer (e.g. glioma), melanoma, prostate cancer, breast cancer, colon cancer, colorectal cancer, lung cancer, kidney cancer, pancreatic cancer, esophageal cancer and bladder cancer.

In related embodiments, a pharmaceutical combination is provided comprising (i) a pseudotyped replicative oncolytic rhabdovirus comprising an arenavirus glycoprotein and (ii) a complement inhibitor. In preferred embodiments, a method for treating and/or preventing cancer and/or treating and/or preventing a metastasis is provided comprising co-administering to a mammal diagnosed with cancer or at risk for developing cancer or a metastasis, (i) a pseudotyped replicative oncolytic rhabdovirus comprising an arenavirus glycoprotein in an amount effective to treat and/or prevent the cancer and/or metastasis and (ii) a complement inhibitor in an amount effective to inhibit complement activity. Preferably the pseudotyped replicative oncolytic rhabdovirus of the combination is administered intratumorally, systemically, particularly intravascularly (intravenously and/or intraarterially), or intracranially and is administered multiple times. In some embodiments, a therapeutic concentration of the pseudotyped replicative oncolytic rhabdovirus is maintained in the mammal for an increased amount of time compared to the same pseudotyped replicative oncolytic rhabodvirus when administered alone (i.e. in the absence of complement inhibitor).

In related embodiments, a method for preventing or reducing the neutralizing effect of antibodies against a replicative oncolytic rhabdovirus pseudotyped with an arenavirus glycoprotein in a mammal is provided comprising co-administering to the mammal one or more complement inhibitors with the pseudotyped replicative oncolytic rhabdovirus. Preferably, the mammal is a human.

In other related embodiments, a method for increasing the persistence of a pseudotyped replicative oncolytic a replicative oncolytic rhabdovirus pseudotyped with an arenavirus glycoprotein in a mammal following one or multiple administrations of said virus to said mammal is provided comprising co-administering to the mammal one or more complement inhibitors with the pseudotyped replicative oncolytic rhabdovirus. Preferably, the mammal is a human.

Complement inhibitors of the combination inhibit, prevent or reduce activation and/or propagation of the complement cascade that results in C3a or signaling through the C3a receptor or formation of C5a or signaling through the C5a receptor. Complement inhibitors useful in the combination include those that operate on one or more of the classical, alternative or lectin pathways. In some embodiments, the complement inhibitor of the combination inhibits the classical pathway. In other embodiments, the complement inhibitor of the combination inhibits the alternative pathway. In yet other embodiments, the complement inhibitor of the combination inhibits the classical and the alternative pathway, in which case the complement inhibitor preferably targets a component of the terminal pathway such as C3 or C5.

Rhabdoviruses of the combination include, without limitation, wild type or genetically modified Arajas virus, Chandipura virus, Cocal virus, Isfahan virus, Maraba virus, Piry virus, Vesicular stomatitis Alagoas virus, BeAn 157575 virus, Boteke virus, Calchaqui virus, Eel virus American, Gray Lodge virus, Jurona virus, Klamath virus, Kwatta virus, La Joya virus, Malpais Spring virus, Mount Elgon bat virus, Perinet virus, Tupaia virus, Farmington, Bahia Grande virus, Muir Springs virus, Reed Ranch virus, Hart Park virus, Flanders virus, Kamese virus, Mosqueiro virus, Mossuril virus, Barur virus, Fukuoka virus, Kern Canyon virus, Nkolbisson virus, Le Dantec virus, Keuraliba virus, Connecticut virus, New Minto virus, Sawgrass virus, Chaco virus, Sena Madureira virus, Timbo virus, Almpiwar virus, Aruac virus, Bangoran virus, Bimbo virus, Bivens Arm virus, Blue crab virus, Charleville virus, Coastal Plains virus, DakArK 7292 virus, Entamoeba virus, Garba virus, Gossas virus, Humpty Doo virus, Joinjakaka virus, Kannamangalam virus, Kolongo virus, Koolpinyah virus, Kotonkon virus, Landjia virus, Manitoba virus, Marco virus, Nasoule virus, Navarro virus, Ngaingan virus, Oak-Vale virus, Obodhiang virus, Oita virus, Ouango virus, Parry Creek virus, Rio Grande cichlid virus, Sandjimba virus, Sigma virus, Sripur virus, Sweetwater Branch virus, Tibrogargan virus, Xiburema virus, Yata virus, Rhode Island, Adelaide River virus, Berrimah virus, Kimberley virus, or Bovine ephemeral fever virus. In some preferred embodiments, the pseudotyped oncolytic rhabdovirus is a pseudotyped wild type or recombinant vesiculovirus. In other preferred embodiments, the pseudotyped oncolytic rhabdovirus of the combination is based on a wild type or recombinant VSV, Farmington, Maraba, Carajas, Muir Springs or Bahia grande virus background strain, including variants thereof. In particularly preferred embodiments, the pseudotyped oncolytic rhabdovirus of the combination is based on a VSV or Maraba rhabdovirus background strain. In other particularly preferred embodiments, the oncolytic rhabdovirus is a VSV or Maraba rhabdovirus comprising one or more genetic modifications that increase tumor selectivity and/or oncolytic effect of the virus.

In related embodiments, the pseudotyped oncolytic rhabdovirus according to the combination therapy is engineered to express one or more tumor antigens, such as those mentioned in paragraphs [0071]-[0082] of WIPO publication no. WO 2014/127478 and paragraph [0042] of U.S. Patent Application Publication No. 2012/0014990. In preferred embodiments, the pseudotyped oncolytic rhabdovirus (e.g. VSV or Maraba strain) expresses MAGEA3, Human Papilloma Virus E6/E7 fusion protein, human Six-Transmembrane Epithelial Antigen of the Prostate protein, or Cancer Testis Antigen 1, or a variant thereof. In particularly preferred embodiments, the oncolytic virus is an oncolytic rhadovirus selected from Maraba and VSVdelta51 that expresses MAGEA3, Human Papilloma Virus E6/E7 fusion protein, human Six-Transmembrane Epithelial Antigen of the Prostate protein, or Cancer Testis Antigen 1, or a variant thereof.

In other embodiments, the pseudotyped oncolytic rhabdovirus according to the combination therapy is engineered to express one or more cytokines.

In other embodiments, one or more immune checkpoint inhibitors are co-administered with the pharmaceutical combination of complement inhibitor and pseudotyped oncolytic rhabdovirus to treat and/or prevent cancer or a metastasis, preferably in a human subject in need thereof.

The pseudotyped oncolytic rhabdovirus of the combination may be administered as one or more doses of 10, 100, 10³, 10⁴, 10⁵, 10⁶, 10⁷, 10⁸, 10⁹, 10¹⁰, 10¹¹, 10¹², 10¹³, 10¹⁴, or more viral particles (vp) or plaque forming units (pfu). In preferred embodiments, the pseudotyped oncolytic rhabdovirus is a wild type or genetically modified VSV or Maraba with an LCMV, Lassa or Junin glycoprotein replacing the VSV or Maraba glycoprotein and optionally expressing one or more tumor antigens and/or cytokines and is administered to a human with cancer as one or more dosages of 10⁶-10¹⁴ pfu, 10⁶-10¹² pfu, 10⁸-10¹⁴ pfu or 10⁸-10¹² pfu. Administration can be by intratumoral, intraperitoneal, intravenous, intra-arterial, intramuscular, intradermal, intracranial, subcutaneous, or intranasal administration. In preferred embodiments, the pseudotyped oncolytic rhabdovirus is administered systemically, particularly by intravascular (intravenous and/or intraarterial) administration, which includes injection, perfusion and the like.

The pseudotyped oncolytic rhabdovirus and complement inhibitor are administered simultaneously or sequentially to the mammal in need thereof and may be administered as part of the same formulation or in different formulations. In some embodiments, a first dose of the complement inhibitor is administered after a first dose of the pseudotyped oncolytic rhabdovirus but prior to a subsequent (e.g. second) dose. In other embodiments, a first dose of the pseudotyped oncolytic rhabodvirus is preceded by a dose of the complement inhibitor and optionally each subsequent dose of the pseudotyped oncolytic rhabdovirus is preceded by a dose of the complement inhibitor. Thus, in some embodiments, a first dose of the complement inhibitor is administered prior to a first dose of the pseudotyped oncolytic rhabdovirus and a second dose of the complement inhibitor is administered prior to a second dose of the pseudotyped oncolytic rhabdovirus and so on.

Cancers to be treated according to the combination described herein include, without limitation, leukemia, acute lymphocytic leukemia, acute myelocytic leukemia, myeloblasts promyelocyte, myelomonocytic monocytic erythroleukemia, chronic leukemia, chronic myelocytic (granulocytic) leukemia, chronic lymphocytic leukemia, mantle cell lymphoma, primary central nervous system lymphoma, Burkitt's lymphoma and marginal zone B cell lymphoma, Polycythemia vera Lymphoma, Hodgkin's disease, non-Hodgkin's disease, multiple myeloma, Waldenstrom's macroglobulinemia, heavy chain disease, solid tumors, sarcomas, and carcinomas, fibrosarcoma, myxosarcoma, liposarcoma, chrondrosarcoma, osteogenic sarcoma, osteosarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon sarcoma, colorectal carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, neuroblastoma, retinoblastoma, nasopharyngeal carcinoma, esophageal carcinoma, basal cell carcinoma, biliary tract cancer, bladder cancer, bone cancer, brain and central nervous system (CNS) cancer, cervical cancer, choriocarcinoma, colorectal cancers, connective tissue cancer, cancer of the digestive system, endometrial cancer, esophageal cancer, eye cancer, head and neck cancer, gastric cancer, intraepithelial neoplasm, kidney cancer, larynx cancer, liver cancer, lung cancer (including small cell lung cancer, squamous non-small cell lung cancer and non-squamous non-small cell lung cancer)), melanoma (including metastatic melanoma), neuroblastoma; oral cavity cancer (for example lip, tongue, mouth and pharynx), ovarian cancer, pancreatic cancer, retinoblastoma, rhabdomyosarcoma, rectal cancer; cancer of the respiratory system, sarcoma, skin cancer, stomach cancer, testicular cancer, thyroid cancer, uterine cancer, and cancer of the urinary system. In some preferred embodiments, the cancer to be treated is selected from non-small cell lung cancer (NSCLC), breast cancer (e.g. hormone refractory metastatic breast cancer), head and neck cancer (e.g. head and neck squamous cell cancer), metastatic colorectal cancer, hormone sensitive or hormone refractory prostate cancer, colorectal cancer, ovarian cancer, hepatocellular cancer, renal cell cancer, soft tissue sarcoma and small cell lung cancer.

In one aspect, the subject to be treated with the combination is a human with a cancer that is refractory to treatment with one or more chemotherapeutic agents and/or refractory to treatment with one or more antibodies.

In a further aspect, the method further comprises administering a chemotherapeutic agent, targeted therapy, radiation, cryotherapy, or hyperthermia therapy to a subject prior to, simultaneously with, or after treatment with the combination therapy.

Related embodiments of the present invention provide a pharmaceutical combination for use in the treatment of cancer or for use in the manufacture of a medicament for treating cancer, in a mammal wherein the combination comprises a pseudotyped oncolytic rhabdovirus, preferably a pseudotyped wild type or attenuated VSV or Maraba virus with an LCMV, Lassa or Junin glycoprotein, and a complement inhibitor. In some embodiments, the pharmaceutical combination comprises a C3 inhibitor and/or a C5 inhibitor and a pseudotyped VSVdelta51 or Maraba virus with an LCMV, Lassa or Junin glycoprotein.

In a further aspect, a kit for use in treating cancer in a mammal is provided including a pseudotyped oncolytic rhabdovirus, preferably a pseudotyped wild type or attenuated Maraba or VSV, and a complement inhibitor. In some embodiments, the kit comprises a VSVdelta51 or Maraba strain rhabdovirus that expresses MAGEA3, a Human Papilloma Virus E6/E7 fusion protein, human Six-Transmembrane Epithelial Antigen of the Prostate Protein, Cancer Testis Antigen 1 or a variant thereof and a complement inhibitor. The kit may further comprise instructions for using the combination for treating cancer.

Methods and compositions of the invention can include a second therapeutic virus, such as an oncolytic or replication defective virus. Oncolytic typically refers to an agent that is capable of killing, lysing, or halting the growth of a cancer cell. In terms of an oncolytic virus the term refers to a virus that can replicate to some degree in a cancer cell, cause the death, lysis, or cessation of cancer cell growth and typically have minimal toxic effects on non-cancer cells. A second virus includes, but is not limited to an adenovirus, a vaccinia virus, a Newcastle disease virus, an alphavirus, a parvovirus, a herpes virus, a rhabdovirus, a non-VSV rhabdovirus and the like. In other aspects, the composition is a pharmaceutically acceptable composition. The composition may also include a second anti-cancer agent, such as a chemotherapeutic, radiotherapeutic, or immunotherapeutic.

Other embodiments of the invention are discussed throughout this application. Any embodiment discussed with respect to one aspect of the invention applies to other aspects of the invention as well, and vice versa. The embodiments in the Detailed Description and Example sections are understood to be non-limiting embodiments of the invention that are applicable to all aspects of the invention.

The terms “inhibiting,” “reducing,” or “preventing,” or any variation of these terms, when used in the claims and/or the specification includes any measurable decrease or complete inhibition to achieve a desired result. Desired results include but are not limited to palliation, reduction, slowing, or eradication of a cancerous or hyperproliferative condition, as well as an improved quality or extension of life.

A “complement inhibitor” is any agent which prevents or reduces the activation of any of the three activation pathways or the terminal pathway. This may ultimately prevent the cleavage of C3 or C5 and the subsequent deposition of associated molecules on the surface of the membrane of the cell or pathogen and release of key signaling molecules. A complement inhibitor can operate on one or more of the complement pathways, i.e., classical, alternative or lectin pathway. A “C3 inhibitor” is a molecule or substance that prevents or reduces the cleavage of C3 into C3a and C3b. A “C5a inhibitor” is a molecule or substance that prevents or reduces the activity of C5a. A “CSaR inhibitor” is a molecule or substance that prevents or reduces the binding of C5a to the C5a receptor. A “C3aR inhibitor” is a molecule or substance that prevents or reduces binding of C3a to the C3a receptor. A “factor D inhibitor” is a molecule or substance that prevents or reduces the activity of Factor D. A “factor B inhibitor” is a molecule or substance that prevents or reduces the activity of factor B. A “C4 inhibitor” is a molecule or substance that prevents or reduces the cleavage of C4 into C4b and C4a. A “C1q inhibitor” is a molecule or substance that prevents or reduces C1q binding to antibody-antigen complexes, virions, infected cells, or other molecules to which C1q binds to initiate complement activation. Any of the complement inhibitors described herein may comprise antibodies or antibody fragments, as would be understood by the person of skill in the art.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

It is to be understood that “combination therapy” envisages the simultaneous, sequential or separate administration of the components of the combination. In one aspect of the invention, “combination therapy” envisages simultaneous administration of the pseudotyped oncolytic rhabdovirus and complement inhibitor. In a further aspect of the invention, “combination therapy” envisages sequential administration of the pseudotyped oncolytic rhabdovirus and complement inhibitor. In another aspect of the invention, “combination therapy” envisages separate administration of the pseudotyped oncolytic rhabdovirus and complement inhibitor. Where the administration of the pseudotyped oncolytic rhabdovirus and complement inhibitor is sequential or separate, the pseudotyped oncolytic rhabdovirus and complement inhibitor are administered within time intervals that allow that the therapeutic agents show a cooperative e.g., synergistic, effect. In preferred embodiments, the pseudotyped oncolytic rhabdovirus and complement inhibitor are administered within 1, 2, 3, 6, 12, 24, 48, 72 hours, or within 4, 5, 6 or 7 days or within 8, 9, 10, 11, 12, 13, 14, 15, 16, 17, 18, 19, 20, 21, 22, 23, 24, 25, 26, 27, 28, 29, 30 or 31 days of each other.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-C. MRB LCMV G antibodies, but not MG1 antibodies, induce virus neutralization only in the presence of complement. FIG. 1A: The in vivo and in vitro treatment schedule. Rats were vaccinated with 10⁷ plaque forming units (pfu) intravenously (IV) of MG1 (Maraba containing G protein Q242R and M protein L123W point mutations) or MRB LCMV G (Maraba virus pseudotyped with LCMV glycoprotein) on Day 0. On Day 15, half the rats from each group were treated with 35 U of cobra venom factor (CVF) to deplete complement. On Day 16, blood was collected from the rats. FIGS. 1B-C: Ex vivo neutralization of MG1 (FIG. 1B) or MRB LCMV G (FIG. 1C) with rat blood, plasma or heat inactivated plasma. Neutralization of MG1 or MRB LCMV G with rat blood, plasma or heat inactivated plasma from unvaccinated (naïve) mice with or without complement depletion is shown for comparison. One rat per immune/complement status was used and data is expressed as the technical replicates ±SD.

FIGS. 2A-B. The complement-dependent antibody neutralization of virus attributed to the LCMV glycoprotein is independent of the rhabdovirus backbone. FIG. 2A: Rats were vaccinated with 10⁸ pfu of MG1 or MRB LCMV G or 10⁷ pfu of wild type Maraba (Maraba wt), VSVd51 or VSV LCMV G (VSV pseudotyped with LCMV glycoprotein). Serum was taken at 14 days post vaccination. Virus neutralization was assessed by ex vivo plaque assay following incubation (1 h; 37° C.) of approximately 5×10⁵ pfu of the corresponding virus with heat inactivated immune serum combined with dextrose gelatin veronal buffer (GVB Control buffer), with naïve rat serum (source of complement) or naïve rat serum pretreated with cobra venom factor (CVF) to depelete C3. FIG. 1B: The relative recovery of input virus is shown for each of the groups. N=2 rats/group; data is expressed as group means±SD.

FIGS. 3A-C. The complement dependent nature of LCMV G pseudotyped rhabdovirus antibody neutralization is not a rodent-specific phenomenon. FIG. 3A: Two cynomolgus macaques received 10¹⁰ pfu intravenously (Animal 1) or 10⁹ pfu intracranially (Animal 2). Neutralization was assessed following ex vivo incubation (1 h; 37° C.) of the heat inactivated immune serum with control buffer, with cynomolgus macaque serum (source of complement) or cynomolgus macaque serum treated with CP40 (complement inhibited). FIG. 3B: Relative recovery of MRB LCMV G virus from Animal 1 immune serum at various time points post vaccination is shown. FIG. 3C: Relative recovery of MRB LCMV G virus from Animal 2 immune serum at various timepoints post vaccination is shown. Data is expressed as the technical replicates ±SD.

FIGS. 4A-D. Abrogating the complement-dependent MRB LCMV G virus neutralization can be accomplished through either the classical or terminal pathway. FIG. 4A: Immune rat serum was collected 18 or 21 days post MRB LCMV G vaccination. The immune serum used in the C3 studies was collected from animals treated with 35 U CVF the day prior to blood draw. The immune rat serum (source of antibody) was combined with control buffer GVB, normal human serum (NHS), C1q immuno-depleted NHS, C3 immuno-depleted NHS, or C5 immuno-depleted NHS (source of complement). Where indicated, C1q or C5 was added back at a concentration of 70 or 75 ug/mL, respectively. Additionally, CP40 was added at a concentration of 25 μM to inhibit human C3 or the C5 monoclonal antibody, eculizumab was used to inhibit C5 at a concentration of 100 μg/mL. MRB LCMV G was incubated with these sources of antibody and complement at 37° C. for 1 h and infectious virus quantified by plaque assay. FIG. 4B: Relative recovery of MRB LCMV G virus from C1q depleted serum. Relative recovery of MRB LCMV G virus from C1q depleted serum (FIG. 4B), from C3 depleted serum (FIG. 4C), and C5 depleted serum (FIG. 4D) is shown.

FIGS. 5A-C. The complement dependence of antibodies generated against surface glycoproteins is a pan-arenavirus phenomenon. FIG. 5A: Rats were vaccinated with 10⁷ plaque forming units (pfu) of MRB Lassa G (Maraba virus pseudotyped with Lassa glycoprotein) or MRB Junin G (Maraba virus pseudotyped with Junin glycoprotein) intravenously and serum taken at 14 days post vaccination. Neutralization was assessed following ex vivo incubation (1 h; 37° C.) of approximately 5×10⁵ pfu of the corresponding virus with heat inactivated immune serum combined with dextrose gelatin veronal buffer (GVB++), with rat serum (source of complement) or rat serum pretreated with CVF to deplete C3 (complement depleted). Recovery of virus was evaluated by plaque assay.

FIG. 5B: Relative recovery of MRB Junin G virus. FIG. 5C: Relative recovery of MRB Lassa G virus. N=3 rats/group; data is expressed as group means±SD.

FIGS. 6A-C. Complement depletion improves the stability and delivery of MRB LCMV G, but not MG1 in immunized animals. FIG. 6A: Fisher rats were vaccinated with MRB LCMV G or MG1 intravenously, or remained virus-naïve. Six days after vaccination, rats were implanted with bilateral 13762 MATBIII tumors. On experiment day 14, half of the animals were depleted of complement with 35 U of CVF. On experiment day 20, the rats were dosed intravenously with the homologous virus at the indicated doses. Rats were sacrificed at 10 minutes after virus treatment, and infectious virus from the blood and tumours was quantified by plaque assay. FIG. 6B: For MRB LCMV G treated animals, infectious virus in the blood and tumours was quantified. FIG. 6C: For MG1 treated animas, infectious virus in the blood and tumours was quantified. N=3 rats per group. Data are represented as group means±SD, Each dot represents a rat. ND=not detected. One way ANOVA (*** p<0.001, **p<0.01, *p<0.05, ^{ns}p>0.05).

FIGS. 7A-C. Complement depletion improved infection of tumors following local administration of MRGB LCMV G but not MG1 in immune rats. FIG. 7A: Fisher rats were vaccinated with MRB LCMV G (FIG. 7B) or MG1 (FIG. 7C) intravenously, or remained virus-naïve. Nine days after vaccination, rats were implanted with bilateral 13762 MATBIII tumors. On experiment day 19, half of the animals were depleted of complement with 35 U of CVF. On experiment day 20, the rats intratumoral injections with the homologous virus at the indicated doses. Rats were sacrificed at 24 hours after virus treatment, and infectious virus from the tumours was quantified by plaque assay. Subcutaneous tumor titers are shown (n=4 per group). Data are represented as group means±SD. Each dot represents a rat. ND=not detected. One way ANOVA (*p<0.05, ^{ns}p>0.05).

FIG. 8. Genome maps of wild type Maraba (Maraba WT), Maraba virus pseudotyped with LCMV glycoprotein (MRB LCMV G), Maraba virus pseudotyped with Junin glycoprotein (MRB LCMV G) and Maraba virus pseudotyped with Lassa glycoprotein (MRB Lassa G).

### DETAILED DESCRIPTION OF THE INVENTION

It has been discovered that replicative oncolytic rhabodviruses pseudotyped with arenavirus glycoproteins elicit an antibody response that requires complement to neutralize viral particles. Furthermore, this neutralization can be prevented by using complement inhibitors or depleting complement, and this leads to increased persistence of infectious virus in the blood.

The present application demonstrates that complement inhibition significantly increases the stability of pseudotyped replicative oncolytic rhabdoviruses in blood and significantly increases delivery of the virus to tumors following local and systemic administration of the virus. Replicative oncolytic rhabdoviruses pseudotyped with arenavirus glycoproteins and their use for treating and/or preventing cancer and/or treating and/or preventing a metastasis in a mammal are provided as well as pharmaceutical combinations comprising (i) an effective amount of replicative oncolytic rhabdovirus pseudotyped with an arenavirus glycoprotein in an amount effective and (ii) a complement inhibitor in an amount effective to inhibit complement activity in the mammal, for use treating and/or preventing cancer and/or treating and/or preventing a metastasis in a mammal.

Embodiments of the invention include compositions and methods related to pseudotyped rhabdoviruses and their use as anti-cancer therapeutics. In particular, pseudotyped rhabdoviruses are provided that are based on a rhabdovirus background strain (or backbone) wherein the glycoprotein gene is substituted for a heterologous arenavirus glycoprotein.

### I. FAMILY RHABDOVIRIDAE (RHABDOVIRUS)

Any replicative oncolytic rhabdovirus strain can be modified to replace the native rhabdovirus glycoprotein with a heterologous arenavirus glycoprotein.

The archetypal rhabdoviruses are rabies and vesicular stomatitis virus (VSV), the most studied of this virus family. Rhabdovirus is a family of bullet shaped viruses having non-segmented (-)sense RNA genomes. The family Rhabdovirus includes, but is not limited to: Arajas virus, Chandipura virus (AF128868/gi:4583436, AJ810083/gi:57833891, AY871800/gi:62861470, AY871799/gi:62861468, AY871798/gi:62861466, AY871797/gi:62861464, AY871796/gi:62861462, AY871795/gi:62861460, AY871794/gi:62861459, AY871793/gi:62861457, AY871792/gi:62861455, AY871791/gi:62861453), Cocal virus (AF045556/gi:2865658), Isfahan virus (AJ810084/gi:57834038), Maraba virus (SEQ ID ON: 1-6 of U.S. Pat. No. 8,481,023, incorporated herein by reference; HQ660076.1), Carajas virus (SEQ ID NO:7-12 of U.S. Pat. No. 8,481,023, incorporated herein by reference, AY335185/gi:33578037), Piry virus (D26175/gi:442480, Z15093/gi:61405), Vesicular stomatitis Alagoas virus, BeAn 157575 virus, Boteke virus, Calchaqui virus, Eel virus American, Gray Lodge virus, Jurona virus, Klamath virus, Kwatta virus, La Joya virus, Malpais Spring virus, Mount Elgon bat virus (DQ457103/gi|91984805), Perinet virus (AY854652/gi:71842381), Tupaia virus (NC_007020/gi:66508427), Farmington, Bahia Grande virus (SEQ ID NO:13-18 of U.S. Pat. No. 8,481,023, incorporated herein by reference, KM205018.1), Muir Springs virus (KM204990.1), Reed Ranch virus, Hart Park virus, Flanders virus (AF523199/gi:25140635, AF523197/gi:25140634, AF523196/gi:25140633, AF523195/gi:25140632, AF523194/gi:25140631, AH012179/gi:25140630), Kamese virus, Mosqueiro virus, Mossuril virus, Barur virus, Fukuoka virus (AY854651/gi:71842379), Kern Canyon virus, Nkolbisson virus, Le Dantec virus (AY854650/gi:71842377), Keuraliba virus, Connecticut virus, New Minto virus, Sawgrass virus, Chaco virus, Sena Madureira virus, Timbo virus, Almpiwar virus (AY854645/gi:71842367), Aruac virus, Bangoran virus, Bimbo virus, Bivens Arm virus, Blue crab virus, Charleville virus, Coastal Plains virus, DakArK 7292 virus, Entamoeba virus, Garba virus, Gossas virus, Humpty Doo virus (AY854643/gi:71842363), Joinjakaka virus, Kannamangalam virus, Kolongo virus (DQ457100/gi|91984799 nucleoprotein (N) mRNA, partial cds); Koolpinyah virus, Kotonkon virus (DQ457099/gi|91984797, AY854638/gi:71842354); Landjia virus, Manitoba virus, Marco virus, Nasoule virus, Navarro virus, Ngaingan virus (AY854649/gi:71842375), Oak-Vale virus (AY854670/gi:71842417), Obodhiang virus (DQ457098/gi|91984795), Oita virus (AB116386/gi:46020027), Ouango virus, Parry Creek virus (AY854647/gi:71842371), Rio Grande cichlid virus, Sandjimba virus (DQ457102/gi|91984803), Sigma virus (AH004209/gi:1680545, AH004208/gi:1680544, AH004206/gi:1680542), Sripur virus, Sweetwater Branch virus, Tibrogargan virus (AY854646/gi:71842369), Xiburema virus, Yata virus, Rhode Island, Adelaide River virus (U10363/gi:600151, AF234998/gi:10443747, AF234534/gi:9971785, AY854635/gi:71842348), Berrimah virus (AY854636/gi:718423501), Kimberley virus (AY854637/gi:71842352), or Bovine ephemeral fever virus (NC_002526/gi:10086561).

In a preferred embodiment, a wild type Maraba strain rhabdovirus or a variant thereof that has optionally been genetically modified e.g. to enhance tumor selectivity serves as the background strain of the pseudotyped oncolytic rhabdovirus. In a particularly preferred embodiment, the psuedotyped oncolytic rhabdovirus is a Maraba strain (e.g. MG1) comprising an arenavirus glycoprotein, preferably an LCMV, Junin or Lassa strain glycoprotein.

In another preferred embodiment, a VSV strain (e.g. VSV Indiana, VSV New Jersey) or a variant thereof that has optionally been genetically modified e.g. to enhance tumor selectivity serves as the background strain of the pseudotyped oncolytic rhabdovirus. In a particularly preferred embodiment, the background strain of the pseudotyped replicative oncolytic rhabdovirus is a VSV comprising a deletion of methionine at position 51 of the M protein (VSVd51) as described in Stojdl et al., Cancer Cell., 4(4):263-75 (2003), the contents of which are incorporated herein by reference. The VSV strain may be further or alternatively attenuated by e.g. mutation and/or deletion of one or more amino acids from the M protein as described in U.S. Pat. No. 8,282,917, the contents of which are hereby incorporated by reference. In some preferred embodiments, the pseudotyped oncolytic rhabodvirus comprises a VSV backbone (e.g. VSVd51) with an LCMV, Junin or Lassa strain glycoprotein.

In other embodiments, the background strain of the pseudotyped replicative oncolytic rhabdovirus comprises genes from two or more strains or serotypes. For example, the background strain may comprise an N, P, M and/or L gene from one strain or serotype and the remaining genes from a different strain or serotype.

Arenavirus Glycoproteins

A (heterologous) glycoprotein from any strain of arenavirus can be substituted into a replicative oncolytic rhabodvirus background to produce a pseudotyped replicative oncolytic virus as herein described, e.g. any of those described in Bowen et al., J. Virology, 6992-7004 (2000). An arenavirus is a virus which is a member of the family Arenaviridae whose members are enveloped viruses with a genome consisting of two single stranded ambisense RNA. The two RNA segments are designated Small (S) and Large (L), each segment coding for two (non-overlapping) viral proteins in opposite orientation. The L segment is approximately 3.5 kb and encodes the viral nucleocapsid protein (NP) and glycoprotein precursor (GPC). The L segment is approximately 7.2 kb and encodes the viral RNA-dependent RNA polymerase (L) and a small RING-domain containing protein (Z). The arenavirus glycoprotein (GP) is a trimeric complex formed by post-translational cleavage of the GPC into the envelope glycoproteins GP1 and GP2 along and a stable signal peptide (SSP) which noncovalently interact to stud the surface of virions.

The arenaviruses have been divided into two serogroups which differ genetically and by geographical distribution—the New World arenaviruses (found in the Eastern Hemisphere) and the Old World arenaviruses (found in the Western Hemisphere). Old World arenaviruses include LCMV, Lassa virus, Mopeia virus, Mobala virus, Ippy virus, Mariental virus, Merino Walk virus, Menekre virus, Gairo virus, Gbagroube virus, Morogoro virus, Kodoko virus, Lunk virus, Okahandja virus, Lujo virus, Lemniscomys virus, Mus minutoides virus, Wenzhou virus, and Luna virus. New World arenaviruses include Tacaribe virus, Junin virus, Machupo virus, Cupixi virus, Amapari virus, Parana virus, Patawa virus, Tamiami virus, Pichinde virus, Latino virus, Flexal virus, Guanarito virus, Sabia virus, Oliveros virus, Whitewater Arroyo virus, Pirital virus, Pampa virus, Bear Canyone virus, Ocozocoautla de Espinosa virus, Allpahuayo virus, Tonto Creek virus, Big Brushy Tank virus, Real de Catorce virus, Catarina virus, Skinner Tank virus, and Chapare virus. In some embodiments, the replicative oncolytic rhabdovirus is pseudotyped with an arenavirus glycoprotein from an Old World complex arenavirus. In other embodiments, the replicative oncolytic rhabdovirus is pseudotyped with an arenavirus glycoprotein from a New World arenavirus.

In some preferred embodiments, the replicative oncolytic rhabdovirus is pseudotyped with an LCMV glycoprotein. LCMV WE strain glycoprotein sequence can be found at GenBank Accession No. AJ297484 and exemplary pHCMV expression vector sequences can be found at Gen Bank Accession Nos. AJ318512 (pHCMV-LCMV-GP(WE)) and AJ318513 (pHCMV-LCMV-GP(WE-HPI)). LCMV Armstrong strain glycoprotein sequence can be found at GenBank Accession No. M20869. In other preferred embodiments, the replicative oncolytic rhabdovirus is pseudotyped with a Lassa glycoprotein. Lassa strain glycoprotein sequences can be found at GenBank Accession No. AAT49014, AAT49012, AAT49010. Examples of DNA sequences encoding Lassa glycoproteins are disclosed under GenBank accession numbers HQ688673 (Josiah segment S, complete sequence), AY179173 (positions 36-1511), AF246121 (positions 54-1529), AF333969 (positions 52-1524), AF181854 (positions 52-1524), and AF181853 (positions 52-1524). In other preferred embodiments, the replicative oncolytic rhabdovirus is pseudotyped with a Junin glycoprotein. An exemplary Junin strain glycoprotein sequence can be found at GenBank Accession No. NC_005081. In some embodiments, the replicative oncolytic rhabdovirus is pseudotyped with an arenavirus glycoprotein that is at least about 70%, 75%, 80%, 85%, 90%, 91%, 92%, 93%, 94%, 95%, 96%, 97%, 98%, or 99% identical to an arenavirus glycoprotein sequence disclosed under GenBank Accession number AJ297484, AAT49014, AAT49012, AAT49010, HQ688673, AY179173, AF246121, AF333969, AF181854, AF181853, or NC_005081.1. In some embodiments, a replicative oncolytic rhabdovirus is pseudotyped with an arenavirus glycoprotein that is not a glycoprotein from an LCMV strain. In other embodiments, a replicative oncolytic rhabdovirus is pseudotyped with an arenavirus glycoprotein that is not a glycoprotein from a Lassa strain. In other embodiments, a replicative oncolytic rhabdovirus is pseudotyped with an arenavirus glycoprotein that is not a glycoprotein from a Lassa strain or a from an LCMV strain. Ippy virus strain glycoprotein sequence can be found at GenBank Accession No. U80003; Mopeia virus strain glycoprotein sequences can be found at GenBank Accession Nos. U80005 (strain AN20410) and M33879 (strain AN21366). Mobala virus train glycoprotein sequence can be found at GenBank Accession No. AF012530 (strain 3076).

In some preferred embodiments, the pseudotyped oncolytic rhabdovirus genome includes the following codon-optimized nucleic acid sequence encoding a Junin strain glycoprotein, an open reading frame thereof or a fragment or variant thereof having at least about 70%, 75%, 80%, 85%, 90%, 91%, 92%, 93%, 94%, 95%, 96%, 97%, 98%, or 99% identity to an open reading frame thereof:

(SEQ ID NO: 1)
GGTACCCAGTTATATTTGTTACAACAATGGGACAATTCATCTCCTTCATG
CAGGAGATACCTACTTTCCTCCAAGAGGCTCTCAATATCGCTCTGGTGGC
GGTTTCACTGATCGCTATCATAAAGGGCATTGTGAACTTGTACAAATCAG
GCCTGTTCCAATTCTTTGTGTTCCTGGCTCTTGCAGGGAGATCTTGTACA
GAAGAGGCTTTTAAAATCGGCCTCCACACTGAGTTTCAGACCGTGAGTTT
CTCAATGGTCGGCCTGTTTTCAAATAATCCCCATGACCTGCCCCTGTTGT
GTACCCTGAACAAGAGTCACCTGTACATCAAGGGCGGAAACGCATCATTC
ATGATCTCCTTTGACGATATTGAAGTGCTGCTGCCTCAATACGATGTGAT
AATACAGCACCCAGCCGACATGTCCTGGTGCAGCAAGTCCGATGACCAAA
TTTGGTTGTCCCAGTGGTTTATGAATGCAGTCGGACATGATTGGCACTTG
GACCCACCCTTCCTTTGCCGCAATAGAACTAAGACCGAGGGTTTCATTTT
TCAGGTCAACACAAGCAAGACTGGGGTCAACGAAAACTATGCAAAAAAGT
TCAAGACAGGTATGCATCACCTCTACCGGGAGTACCCTGATTCTTGCCTG
AACGGGAAGTTGTGCCTGATGAAGGCCCAGCCAACGTCCTGGCCTCTGCA
GTGCCCTTTGGACCATGTGAACACTTTGCACTTTCTCACTAGAGGCAAAA
ACATCCAGCTCCCTAGGCGATCCCTTAAGGCGTTCTTTTCTTGGAGTCTG
ACGGATTCTTCCGGAAAGGACACCCCTGGGGGCTACTGTCTCGAAGAATG
GATGCTGGTAGCTGCAAAGATGAAATGTTTTGGGAACACTGCCGTCGCGA
AATGCAACCTGAACCATGATTCTGAATTTTGCGATATGCTCCGACTTTTC
GACTATAATAAGAATGCTATCAAGACACTGAACGATGAAACTAAGAAACA
GGTGAATCTCATGGGACAGACCATTAATGCTCTGATCAGTGACAATCTGC
TGATGAAGAATAAAATCCGAGAGCTGATGTCAGTGCCCTATTGTAATTAT
ACAAAATTTTGGTACGTGAATCACACACTGTCCGGCCAGCACTCTCTGCC
GAGGTGCTGGCTGATTAAGAATAATAGCTACTTGAACATCAGCGACTTCA
GAAACGACTGGATTCTCGAGTCCGATTTTCTGATCAGCGAAATGCTCAGT
AAAGAGTATTCAGACAGACAGGGCAAGACACCCCTTACTCTCGTTGATAT
TTGTTTTTGGAGTACAGTTTTTTTTACGGCCTCCCTGTTCCTCCATCTGG
TCGGTATTCCTACCCACCGACATATCCGCGGCGAGGCATGTCCACTGCCT
CATCGCCTCAATTCACTGGGAGGCTGTCGATGTGGAAAGTATCCGAATCT
CAAAAAACCTACCGTCTGGCGCAGAAGACATTAGGCGGCCGC

In related embodiments, the pseudotyped oncolytic rhabdovirus genome comprises a nucleic acid sequence encoding the following Junin glycoprotein or a functional fragment or variant thereof having at least about 70%, 75%, 80%, 85%, 90%, 91%, 92%, 93%, 94%, 95%, 96%, 97%, 98%, or 99% identity thereto:

(SEQ ID NO: 2)
MGQFISFMQEIPTFLQEALNIALVAVSLIAIIKGIVNLYKSGLFQFFVFL
ALAGRSCTEEAFKIGLHTEFQTVSFSMVGLFSNNPHDLPLLCTLNKSHLY
IKGGNASFMISFDDIEVLLPQYDVIIQHPADMSWCSKSDDQIWLSQWFMN
AVGHDWHLDPPFLCRNRTKTEGFIFQVNTSKTGVNENYAKKFKTGMHHLY
REYPDSCLNGKLCLMKAQPTSWPLQCPLDHVNTLHFLTRGKNIQLPRRSL
KAFFSWSLTDSSGKDTPGGYCLEEWMLVAAKMKCFGNTAVAKCNLNHDSE
FCDMLRLFDYNKNAIKTLNDETKKQVNLMGQTINALISDNLLMKNKIREL
MSVPYCNYTKFWYVNHTLSGQHSLPRCWLIKNNSYLNISDFRNDWILESD
FLISEMLSKEYSDRQGKTPLTLVDICFWSTVFFTASLFLHLVGIPTHRHI
RGEACPLPHRLNSLGGCRCGKYPNLKKPTVWRRRH

In some preferred embodiments, the pseudotyped oncolytic rhabdovirus genome includes the following codon-optimized nucleic acid sequence encoding a Lassa strain glycoprotein, an open reading frame thereof or a fragment or variant thereof having at least about 70%, 75%, 80%, 85%, 90%, 91%, 92%, 93%, 94%, 95%, 96%, 97%, 98%, or 99% identity to an open reading frame thereof:

(SEQ ID NO: 3)
GGTACCCAGTTATATTTGTTACAACAATGGGACAAATCATCACGTTTTTC
CAGGAAGTGCCCCACGTCATAGAGGAGGTAATGAATATAGTGCTCATTGC
CCTCAGTTTGCTGGCGATCCTGAAAGGGATCTACAACGTGGCGACTTGTG
GTCTGTTTGGCTTGGTGTCTTTCCTGCTGTTGTGCGGTCGAAGCTGCAGT
ACCACCTATAAGGGAGTCTACGAGCTGCAGACACTGGAACTGGACATGGC
TAGCTTGAACATGACTATGCCTCTCTCCTGCACAAAGAATAACAGTCACC
ATTACATAATGGTGGGGAATGAAACTGGTTTGGAACTCACACTTACCAAC
ACATCCATCATAAATCACAAGTTTTGTAACCTCAGTGACGCCCACAAAAA
AAACTTGTATGATCACGCTCTCATGTCCATAATCAGCACTTTTCACCTGT
CTATCCCTAACTTCAATCAGTACGAGGCTATGTCTTGCGACTTTAACGGG
GGCAAAATCAGCGTGCAATACAATCTGAGCCACGCATATGCCGTCGACGC
CGCCAACCACTGCGGAACTATCGCTAACGGCGTCCTGCAGACATTCATGC
GGATGGCTTGGGGCGGCTCCTATATCGCTCTGGATAGCGGAAAGGGCAGT
TGGGACTGTATTATGACCTCATACCAGTACCTTATTATCCAGAACACCAC
CTGGGAGGATCACTGTCAATTTTCCCGGCCGTCCCCAATCGGCTATCTGG
GCCTCCTGAGCCAAAGAACTCGGGACATTTACATATCTCGGCGACTCCTC
GGGACATTCACATGGACCCTGTCCGACTCTGAAGGGAATGAAACGCCAGG
CGGGTATTGCCTGACCCGATGGATGCTGATCGAAGCCGAGCTCAAGTGCT
TTGGAAATACCGCAGTCGCCAAGTGTAATGAAAAGCATGATGAAGAATTT
TGCGATATGCTGCGGCTGTTCGATTTCAATAAACAGGCCATTCGACGGCT
GAAAACCGAGGCCCAAATGAGTATCCAGCTGATTAACAAGGCCGTTAATG
CCCTGATTAATGACCAGCTCATTATGAAAAATCACCTGCGGGATATCATG
GGCATTCCTTACTGTAACTATTCCAAGTATTGGTATCTGAACCACACCGT
GACTGGCAAAACGTCACTGCCAAGGTGCTGGCTGGTCTCCAATGGAAGCT
ACCTGAACGAGACCCATTTTTCCGATGATATCGAGCAGCAGGCCGATAAT
ATGATTACCGAACTGTTGCAGAAAGAATACATGGACCGCCAGGGCAAAAC
TCCACTTGGGTTGGTCGACCTGTTTGTGTTCTCTACCAGCTTCTACTTGA
TTAGCATTTTCCTGCACCTGGTGCGCATCCCCACGCACAGACATGTCATC
GGTAAGCCATGCCCTAAGCCGCATAGACTCAACCATATGGGGATTTGCTC
CTGTGGTCTCTATAAACACCCCGGCGTGCCTGTCAAATGGAAGAGGTGAG
CGGCCGC

In related embodiments, the pseudotyped oncolytic rhabdovirus genome comprises a nucleic acid sequence encoding the following Lassa glycoprotein or a functional fragment or variant thereof having at least about 70%, 75%, 80%, 85%, 90%, 91%, 92%, 93%, 94%, 95%, 96%, 97%, 98%, or 99% identity thereto:

(SEQ ID NO: 4)
MGQIITFFQEVPHVIEEVMNIVLIALSLLAILKGIYNVATCGLFGLVSFL
LLCGRSCSTTYKGVYELQTLELDMASLNMTMPLSCTKNNSHHYIMVGNET
GLELTLTNTSIINHKFCNLSDAHKKNLYDHALMSIISTFHLSIPNFNQYE
AMSCDFNGGKISVQYNLSHAYAVDAANHCGTIANGVLQTFMRMAWGGSYI
ALDSGKGSWDCIMTSYQYLIIQNTTWEDHCQFSRPSPIGYLGLLSQRTRD
IYISRRLLGTFTWTLSDSEGNETPGGYCLTRWMLIEAELKCFGNTAVAKC
NEKHDEEFCDMLRLFDFNKQAIRRLKTEAQMSIQLINKAVNALINDQLIM
KNHLRDIMGIPYCNYSKYWYLNHTVTGKTSLPRCWLVSNGSYLNETHFSD
DIEQQADNMITELLQKEYMDRQGKTPLGLVDLFVFSTSFYLISIFLHLVR
IPTHRHVIGKPCPKPHRLNHMGICSCGLYKHPGVPVKWKR

Preferably, the pseudotyped virus's genome or plasmid encoding the pseudotyped virus's genome encodes the entire arenavirus glycoprotein precursor, such that both GP1 and GP2 are expressed and contribute to formation of the pseudotyped virus's envelope. In other embodiments, the pseudotyped virus's genome or plasmid encoding the pseudotyped virus's genome encodes less than the entire arenavirus glycoprotein precusor. For example, in embodiments, the pseudotyped virus's genome or plasmid encoding the recombinant viral genome encodes a truncated GPC or only GP1 or only GP2.

Additional Heterologous Nucleic Acid Sequences

In other preferred embodiments, the pseudotyped oncolytic rhabdovirus expresses one or more tumor antigens such as oncofetal antigens such as alphafetoprotein (AFP) and carcinoembryonic antigen (CEA), surface glycoproteins such as CA 125, oncogenes such as Her2, melanoma-associated antigens such as dopachrome tautomerase (DCT), GP100 and MART1, cancer-testes antigens such as the MAGE proteins and NY-ESO1, viral oncogenes such as HPV E6 and E7, and proteins ectopically expressed in tumours that are usually restricted to embryonic or extraembryonic tissues such as PLAC or a variant of a tumor-associated antigen. A “variant” of a tumor associated antigen refers to a protein that (a) includes at least one tumor associated antigenic epitope from the tumor associated antigenic protein and (b) is at least 70%, preferably at least 80%, more preferably at least 90% or at least 95% identical to the tumor associated antigenic protein. A database summarizing well accepted antigenic epitopes is provided by Van der Bruggen P, Stroobant V, Vigneron N, Van den Eynde B in “Database of T cell-defined human tumor antigens: the 2013 update.” Cancer Immun 2013 13:15 and www.cancerimmunity.org/peptide. In particularly preferred embodiments, the pseudotyped oncolytic rhadovirus expresses MAGEA3, Human Papilloma Virus E6/E7 fusion protein, human Six-Transmembrane Epithelial Antigen of the Prostate protein, or Cancer Testis Antigen 1. In related aspects, a pseudotyped oncolytic rhabdovirus expressing a tumor antigen is co-administered with a complement inhibitor to a mammal with cancer to treat the cancer. The mammal may have a pre-existing immunity to the tumor antigen that is naturally existing or that is established by administering the tumor antigen to the mammal prior to administering the pseudotyped oncolytic rhabdovirus expressing the tumor antigen.

The MAGE family of genes encoding tumor specific antigens is discussed in De Plaen et al., Immunogenetics 40:360-369 (1994). MAGEA3 is expressed in a wide variety of tumours including melanoma, non-small cell lung cancer, head and neck cancer, colorectal cancer and bladder cancer. Tumor associated antigenic epitopes have been already identified for MAGEA3 and any of these epitopes may be expressed by the pseudotyped oncolytic rhabdovirus.

Human Papilloma Virus (HPV) oncoproteins E6/E7 are constitutively expressed in cervical cancer (Zur Hausen, H (1996) Biochem Biophys Acta 1288:F55-F78). Furthermore, HPV types 16 and 18 are the cause of 75% of cervical cancer (Walboomers J M (1999) J Pathol 189: 12-19).

Six-Transmembrane Epithelial Antigen of the Prostate (huSTEAP) is a recently identified protein shown to be overexpressed in prostate cancer and up-regulated in multiple cancer cell lines, including pancreas, colon, breast, testicular, cervical, bladder, ovarian, acute lyphocytic leukemia and Ewing sarcoma (Hubert R S et al., (1999) Proc Natl Acad Sci 96: 14523-14528). The STEAP gene encodes a protein with six potential membrane-spanning regions flanked by hydrophilic amino- and carboxyl-terminal domains.

Cancer Testis Antigen 1 (NYES01) is a cancer/testis antigen expressed in normal adult tissues, such as testis and ovary, and in various cancers (Nicholaou T et al., (2006) Immunol Cell Biol 84:303-317). Cancer testis antigens are a unique family of antigens, which have restricted expression to testicular germ cells in a normal adult but are aberrantly expressed on a variety of solid tumours, including soft tissue sarcomas, melanoma and epithelial cancers.

In other embodiments, a pseudotyped oncolytic rhabdovirus expresses one or more cytokines such as granulocyte macrophage colony stimulating factor (GM-CSF), tumor necrosis factor alpha (TNFα), tumor necrosis factor beta (TNFβ), interleukin 1 (IL-1), interleukin 2 (IL-2), interleukin 4 (IL-4), interleukin 5 (IL-5), interleukin 6 (IL-6), interleukin 10 (IL-10), interleukin 12 (IL-12), interleukin 15 (IL-15), interleukin 21 (IL-21), interferon alpha (IFNα), interferon beta (IFNβ), interferon gamma (IFNγ) and variants and fragments thereof. In related aspects, a pseudotyped oncolytic rhabdovirus expressing a cytokine is co-administered with a complement inhibitor to a mammal with cancer to treat the cancer. In other embodiments, a pseudotyped oncolytic rhabdovirus expresses one or more immune checkpoint inhibitors that bind to and antagonize the activity of an immune checkpoint protein such as cytotoxic T-lymphocyte antigen-4 (CTLA4), programmed cell death protein 1 (PD-1) and its ligands PD-L1 and PD-L2, B7-H3, B7-H4, herpesvirus entry mediator (HVEM), T cell membrane protein 3 (TIM3), galectin 9 (GALS), lymphocyte activation gene 3 (LAG3), V-domain immunoglobulin (Ig)-containing suppressor of T-cell activation (VISTA), Killer-Cell Immunoglobulin-Like Receptor (KIR), B and T lymphocyte attenuator (BTLA), T cell immunoreceptor with Ig and ITIM domains (TIGIT) or a combination thereof. In preferred embodiments, the immune checkpoint inhibitor is an anti-PD-1, anti-PD-L1, or anti-CLTA4 antibody or antigen-binding fragment thereof or a fusion protein. In some embodiments, the pseudotyped oncolytic rhabdovirus expresses a monoclonal antibody against CTLA4 such as Ipilimumab (Yervoy®; BMS) or Tremelimumab (AstraZeneca/MedImmune) and/or a monoclonal antibody against PD-1 such as Nivolumab (Opdivo®; Bristol-Myers Squibb; code name BMS-936558), Pembrolizumab (Keytrudaθ) or Pidilizumab.

Routes of administration of the pesudotyped oncolytic rhabdovirus according to the methods herein described will vary, naturally, with the location and nature of the lesion, and include, e.g., intradermal, transdermal, parenteral, intravascular (intravenous or intraarterial), intramuscular, intranasal, subcutaneous, regional, percutaneous, intratracheal, intraperitoneal, intravesical, intratumoral, inhalation, perfusion, lavage, direct injection, alimentary, and oral administration and formulation. In preferred embodiments, a pharmaceutical composition comprising the pseudotyped oncolytic rhabdovirus of the combination and a pharmaceutically acceptable carrier is administered to a mammal with cancer by intratumoral injection and/or is administered intravascularly, although the pharmaceutical composition may alternatively be administered intratumorally, parenterally, intravenously, intrarterially, intradermally, intramuscularly, transdermally, intracranially or even intraperitoneally as described in U.S. Pat. Nos. 5,543,158, 5,641,515 and 5,399,363 (each specifically incorporated herein by reference in its entirety). As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

In certain embodiments, the tumor being treated may not, at least initially, be resectable. Treatments with therapeutic viral constructs may increase the resectability of the tumor due to shrinkage at the margins or by elimination of certain particularly invasive portions. Following treatments, resection may be possible. Additional treatments subsequent to resection will serve to eliminate microscopic residual disease at the tumor site.

A typical course of treatment, for a primary tumor or a post-excision tumor bed, will involve multiple doses. Typical primary tumor treatment involves a 1, 2, 3, 4, 5, 6 or more dose application over a 1, 2, 3, 4, 5, 6-week period or more. A two-week regimen may be repeated one, two, three, four, five, six or more times. During a course of treatment, the need to complete the planned dosings may be re-evaluated. In some embodiments, when co-administered with a complement inhibitor, a second, third, fourth, fifth, sixth or subsequent administration of a pseudotyped oncolytic rhabdovirus occurs without a substantial decrease in efficacy and/or without a substantial increase in dose relative to a previously administered dose.

The treatments may include various “unit doses.” Unit dose is defined as containing a predetermined quantity of the therapeutic composition. The quantity to be administered, and the particular route and formulation, are within the skill of those in the clinical arts. A unit dose need not be administered as a single injection but may comprise continuous infusion over a set period of time. Unit dose of the present invention may conveniently be described in terms of plaque forming units (pfu) or viral particles for viral constructs. Unit doses range from 10³, 10⁴, 10⁵, 10⁶, 10⁷, 10⁸, 10⁹, 10¹⁰, 10¹¹, 10¹², 10¹³ pfu or vp and higher. Alternatively, depending on the kind of virus and the titer attainable, one will deliver 1 to 100, 10 to 50, 100-1000, or up to about 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, or 1×10¹⁵ or higher infectious viral particles (vp) to the patient or to the patient's cells.

The phrase “pharmaceutically-acceptable” or “pharmacologically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

### II. COMPLEMENT INHIBITORS

In various aspects, a combination therapy for treating and/or preventing cancer and/or treating and/or preventing a metastasis is provided comprising co-administering to a mammal in need thereof (i) a replicative oncolytic rhadovirus pseudotyped with an arenavirus glycoprotein and (ii) one or more complement inhibitors, in combined amounts effective to treat and/or prevent the cancer.

The complement system is a key component of innate immunity. The complement system can be activated by any one of three separate pathways—the classical pathway. the alternative pathway, and the lectin pathway, all of which differ in their mode of recognition but converge in the generation of C3 convertases that cleave the central component C3 to C3a and C3b. Subsequently, the C3 convertase is changed into a C5 convertase by inclusion of another C3b molecule to the C3 convertase. This C5 convertase cleaves C5 resulting in release of C5a and formation of the C5b-9 complex. Complement inhibitors prevent or reduce activation and/or propagation of the complement cascade that results in C3a or signaling through the C3a receptor or formation of C5a or signaling through the C5a receptor. Complement inhibitors useful in the combination include those that operate on one or more of the classical, alternative or lectin pathways, or on the shared terminal pathway.

Inhibitors that target C3 can inhibit all three (classical, alternative and lectin) pathways due to the central position of C3 in the complement activation process. In some embodiments, the complement inhibitor of the combination inhibits C3. Complement inhibitors that inhibit C3 include without limitation the humanized monoclonal antibody H17 (EluSys Therapeutics), the cyclic peptide compstatin and analogs, peptidomimetics, and derivatives thereof such as 4(1MeW)/POT-4 (Potentia), 4(1MeW)/APL-1, APL-2 (Apellis), Cp40/AMY-101, PEG-Cp40 (Amyndas), as well those described in U.S. Pat. Nos. 6,319,897 and 7,888,323 and WIPO Publication No. WO 2013/036778A2, the contents of each of which are incorporated herein by reference, CFH-based proteins such as TT30 (CR2/CFH; Alexion), MiniCFH (Amyndas), CR1-based proteins such as sCR1 (CDX-1135; Celldex/Avant Immunotherapeutics), Microcept (APT070) and TT32 (CR2/CR1; Alexion Pharmaceuticals). In a preferred embodiment, the complement inhibitor is compstatin or an analog, peptidomimetic or derivative thereof.

Inhibitors that target C5 can also inhibit all three pathways. Thus, in other embodiments, the complement inhibitor of the combination inhibits complement component 5 (C5). Complement inhibitors that target C5 include, without limitation, monoclonal antibodies such as Eculizumab (Soliris; Alexion Pharmaceuticals) and LFG316 (Novartis/Morphosys), human minibodies such as Mubodina (Adienne), humanized single chain variable fragments (scFVs) such as Pexelizumab (Alexion Pharmaceuticals), recombinant proteins such as Coversin (OmCl; Volution Immuno-Pharmaceuticals), aptamers such as ARC1005 (NovoNordisk), ARC1905 (Ophthotech) and SOMAmers (SomaLogic), affibodies such as SOB1002 (fused with albumin-binding domain; Swedish Orpahn Biovitrum), siRNAs such as Anti-05 siRNA (Alnylam). In a preferred embodiment, the complement inhibitor is eculizumab, a humanized monoclonal antibody that binds to C5 and inhibits its cleavage to C5a and C5b.

The classical pathway is activated by the formation of antigen-antibody complexes. C1, the first enzyme complex in the cascade, consists of C1q, 2 C1r molecules and 2 C1s molecules. This complex binds to antigen-antibody complex through the C1q domain to initiate the cascade. Once activated, C1s cleaves C4 resulting in C4b, which in turn binds C2. C2 is cleaved by C1 resulting in the activated form, C2a, bound to C4b (C4b2a) and forming the classical pathway C3 convertase. C4b2a is subsequently transformed into a C5 convertase by the binding of an additional C3b molecule. The classical pathway can be specifically inhibited e.g. by targeting C2a and/or the C2a portion of C2. In one aspect, an inhibitor of the classical pathway is a monoclonal anti-C2a antibody that interfere with the interaction between C2 and C4. The classical pathway can be specifically inhibited e.g. by targeting C2a and/or the C2a portion of C2. In one aspect, an inhibitor of the classical pathway is a monoclonal anti-C2a antibody that interferes with the interaction between C2 and C4. In other embodiments, the complement inhibitor inhibits C1. Complement inhibitors that inhibit C1 (e.g. C1s) include without limitation purified or recombinant C1 esterase inhibitor (e.g. Cinryze (ViroPharma/Baxter)) and monoclonal antibodies such as TNT003, TNT009 and TNT010 (True North Therapeutics). In a preferred embodiment, the complement inhibitor is Cinryze, TNT009 or TNT010. Factor I also inhibits the classical pathway by inhibiting the classical C3 convertase.

The alternative pathway (AP) lacks a specific recognition molecule. In this pathway, the assembly of C3 convertases is initiated by covalent attachment of C3b to the activator surface. In the next step, complement factor B (CFB) binds to surface-bound C3b and is subsequently cleaved by complement factor D (CFD), generating C3bBb. C3bBb is subsequently transformed into a C5 convertase by the binding of an additional C3b molecule. In some embodiments, the complement inhibitor inhibits CFB and/or CFD. Complement inhibitors that inhibit CFB include monoclonal antibodies such as TA106 (Alexion Pharmaceuticals) and siRNAs such as Anti-FB siRNA (Alnylam). Complement inhibitors that inhibit CFD include monoclonal antibodies such as FCFD4514S (Genentech/Roche) and antigen binding antibody fragments such as lampalizumab (Genetech). Complement inhibitors that inhibit CFD and CFB include aptamers such as SOMAmers (SomaLogic) and small molecule inhibitors such as those available from Novartis. Factor H (inactive C3b), a soluble glycoprotein, also inhibits the alternative pathway by inhibiting the formation of the C3 convertase by competing with factor B for binding to C3b.

Other examples of agents that inhibit complement biological activity include, but are not limited to: C5a receptor antagonists, for example, NGD 2000-1 (Neurogen, Corp., Branford, Conn.), CCX168 (ChemoCentryx), PMX53 (Promics/Cephalon) and AcPhe[Orn-Pro-D-Cyclohexylalanine-Trp-Arg] (AcF-[OPdChaWR]; see, e.g., Strachan, A. J. et al., Br. J. Pharmacol. 134(8):1778-1786 (2001)); Factor I (inactive C4b); soluble complement receptor type 1 (sCR1; see, e.g., U.S. Pat. No. 5,856,297) and sCR1-sLe(X) (see, e.g., U.S. Pat. No. 5,856,300; membrane cofactor protein (MCP), decay accelerating factor (DAF) and CD59 and soluble recombinant forms thereof (Ashgar, S. S. et al., Front Biosci. 5:E63-E81 (2000) and Sohn, J. H. et al., Invest. Opthamol. Vis. Sci. 41(13):4195-4202 (2000)); chimeric complement inhibitor proteins having at least two complementary inhibitory domains (see, e.g., U.S. Pat. Nos. 5,679,546, 5,851,528 and 5,627,264); and small molecule antagonists (see, e.g., PCT Publication No. WO 02/49993, U.S. Pat. Nos. 5,656,659, 5,652,237, 4,510,158, 4,599,203 and 4,231,958). Other known complement inhibitors are known in the art and are encompassed by the methods herein described. In addition, methods for measuring complement activity (e.g., to identify agents that inhibit complement activity) are known in the art. Such methods include, e.g., using a 50% hemolytic complement (CH₅₀) assay (see, e.g., Kabat et al., Experimental Immunochemistry, 2nd Ed. (Charles C. Thomas, Publisher, Springfield, Ill.), p. 133-239 (1961)), using an enzyme immunoassay (EIA), using a liposome immunoassay (LIA) (see, e.g., Jaskowski et al., Clin. Diagn. Lab. Immunol. 6(1):137-139 (1999)).

Complement inhibitors according to the combination can be administered to a mammal with cancer by any suitable administration route, including intravascular (intravenous and/or intraarterial), intramuscular, subcutaneous, intravitreal, and oral.

Complement inhibitors according to the combination are co-administered to a mammal with pseudotyped replicative oncolytic rhabdovirus in a combined amount that is effective to treat and/or prevent cancer in the mammal. Typically, a complement inhibitor is administered in an amount effective to inhibit complement activity in the mammal. Appropriate dosages are known in the art and depend on the inhibitor being administered. For antibodies, appropriate dosages generally range from 0.1 mg/kg and 20 mg/kg of the patient's body weight, preferably between 1 mg/kg and 10 mg/kg of the patient's body weight. For example, eculizumab can be administered by intravenous infusion at a dose of 600 or 900 mg every 7 days for 1, 2, 3, 4 or more weeks, after which the dose can be increased to 900 or 1200 mg administered once (7 days later) and then 900 or 1200 mg every two weeks thereafter. Pexelizumab can be administered by a single 2.0 mg/kg bolus optionally followed by 0.05 mg/kg/hr infusion for 20 to 24 hours. Cinryze can be administered as 1000 U intravenously every 3 or 4 days. The peptide compstatin and its analogs (e.g. CP40) can be administered at one (e.g. a single bolus) or more dosages of e.g. between about 0.5 mg/kg and 25 mg/kg. For example, compstatin or an analog thereof may be administered as a single bolus of e.g. 2-10 mg/kg optionally followed by continuous infusion.

In some embodiments, a single complement inhibitor is co-administered to mammal with a pseudotyped replicative oncolytic rhabdovirus to treat and/or prevent cancer. In other embodiments, a combination of two or more complement inhibitors are co-administered with a pseudotyped replicative oncolytic rhabdovirus to treat and/or prevent cancer. For example, a combination of an inhibitor of the classical pathway and an inhibitor of the alternative pathway can be co-administered with a pseudotyped replicative oncolytic rhabodvirus to a mammal in order to treat and/or prevent cancer in the mammal.

Additional Therapeutics

The compounds and methods of the present invention may be used in the context of cancer. In order to increase the effectiveness of the treatment methods described herein, it may be desirable to combine compositions as described herein with other agents effective in the prevention/treatment of cancer. For example, the treatment of a cancer may be implemented with therapeutic compounds of the present invention and other anti-cancer therapies, such as anti-cancer agents or surgery.

An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. Anti-cancer agents include biological agents (biotherapy), chemotherapy agents, and radiotherapy agents. More generally, these other compositions would be provided in a combined amount effective to kill or inhibit proliferation of the cell. This process may involve contacting the cells with virus or viral construct and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the virus and the other includes the second agent(s).

Tumor cell resistance to chemotherapy and radiotherapy agents represents a major problem in clinical oncology. One goal of current cancer research is to find ways to improve the efficacy of chemo- and radiotherapy by combining it with gene therapy. For example, the herpes simplex-thymidine kinase (HS-tK) gene, when delivered to brain tumors by a retroviral vector system, successfully induced susceptibility to the antiviral agent ganciclovir (Culver et al., 1992). In the context of the present invention, it is contemplated that poxvirus therapy could be used similarly in conjunction with chemotherapeutic, radiotherapeutic, immunotherapeutic, or other biological intervention, in addition to other pro-apoptotic or cell cycle regulating agents.

Alternatively, a viral therapy may precede or follow the other treatment by intervals ranging from minutes to weeks. In embodiments where the other agent and virus are applied separately to the cell, one would generally ensure that a significant period of time did not expire between the time of each delivery, such that the agent and virus would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein transferase inhibitors, transplatinum, 5-fluorouracil, vincristine, vinblastine and methotrexate, Temazolomide (an aqueous form of DTIC), or any analog or derivative variant of the foregoing. The combination of chemotherapy with biological therapy is known as biochemotherapy.

Other factors that cause DNA damage and have been used extensively include what are commonly known as γ-rays, X-rays, proton beams, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

The proteins that induce cellular proliferation further fall into various categories dependent on function. The commonality of all of these proteins is their ability to regulate cellular proliferation. For example, a form of PDGF, the sis oncogene, is a secreted growth factor. Oncogenes rarely arise from genes encoding growth factors, and at the present, the sis oncogene is the only known naturally-occurring oncogenic growth factor. In one embodiment of the present invention, it is contemplated that anti-sense mRNA directed to a particular inducer of cellular proliferation is used to prevent expression of the inducer of cellular proliferation.

The tumor suppressor oncogenes function to inhibit excessive cellular proliferation. The inactivation of these genes destroys their inhibitory activity, resulting in unregulated proliferation. Tumor suppressors include p53, p16 and C-CAM. Other genes that may be employed according to the present invention include Rb, APC, DCC, NF-1, NF-2, WT-1, MEN-I, MEN-II, zacl, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, erb, fms, trk, ret, gsp, hst, abl, E1A, p300, genes involved in angiogenesis (e.g., VEGF, FGF, thrombospondin, BAI-1, GDAIF, or their receptors) and MCC.

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl 2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl 2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl 2 (e.g., BclXL, BclW, BclS, Mcl-1, A1, Bfl-1) or counteract Bcl 2 function and promote cell death (e.g., Bax, Bak, Bik, Bim, Bid, Bad, Harakiri).

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, pre-cancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

It is contemplated that other agents may be used in combination with the compositions and methods described herein to improve the therapeutic efficacy of treatment. These additional agents include immunomodulatory agents such as the immune checkpoint inhibitors described above, agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Immunomodulatory agents include tumor necrosis factor; interferon α, β, and γ; IL-2 and other cytokines; F42K and other cytokine analogs; or MIP-1, MIP-1β, MCP-1, RANTES, and other chemokines. It is further contemplated that the upregulation of cell surface receptors or their ligands such as Fas/Fas ligand, DR4 or DR5/TRAIL (Apo-2 ligand) would potentiate the apoptotic inducing ability of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

There have been many advances in the therapy of cancer following the introduction of cytotoxic chemotherapeutic drugs. However, one of the consequences of chemotherapy is the development/acquisition of drug-resistant phenotypes and the development of multiple drug resistance. The development of drug resistance remains a major obstacle in the treatment of such tumors and therefore, there is an obvious need for alternative approaches such as viral therapy.

Another form of therapy for use in conjunction with chemotherapy, radiation therapy or biological therapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment.

### III. EXAMPLES

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1

Methods

Viruses and Cells.

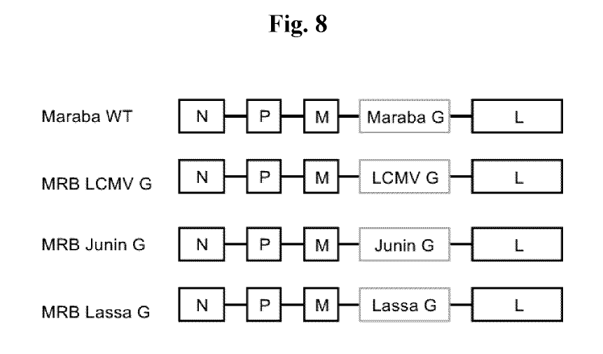

Vero, 13762 MAT B III and 9L/LacZ cells were purchased from the American Type Culture Collection (Manassas, Va.). 13762 MAT B III cells were maintained in McCoy's 5A (ATCC, Manassas, Va.) supplemented with 10% fetal bovine serum (HyClone, Logan, Utah). 9L/LacZ and Vero cells were maintained in Dulbecco's Modified Eagle's medium (HyClone, Logan, Utah) supplemented with 10% fetal bovine serum (HyClone, Logan, Utah). Maraba MG1 (Maraba containing G protein Q242R and M protein L123W point mutations) and Maraba wild type were used as previously described. VSVd51 (VSV containing a deletion of methionine at position 51 of the M protein) and VSV LCMV G (VSV comprising a gene encoding glycoprotein (G) of LCMV and lacking a functional gene coding for envelope protein G of VSV) were used as previously described. MRB LCMV G (or MV-LCMVg) was produced by replacing the G protein of Maraba with a gene encoding glycoprotein (G) of LCMV. MRB Lassa G (or MV-Lg) was produced by replacing the G protein of Maraba with a gene encoding glycoprotein (G) of Lassa virus. MRB Junin G (or MV-Jg) was produced by replacing the G protein of Maraba with a gene encoding glycoprotein (G) of Junin virus. See FIG. 8. In each of the pseudotyped viruses, the native VSV or Maraba G protein was deleted and a codon-optimized gene encoding LCMV, Junin or Lassa virus glycoprotein was substituted in the same position from which the VSV or Maraba G protein gene had been deleted. Briefly, codon optimized genes encoding Junin glycoprotein and Lassa glycoprotein were synthesized and engineered with an upstream KpnI restriction site and downstream NotI restriction site. KpnI/NotI Junin glycoprotein and Lassa glycoprotein fragments were cloned into the NotI-glycoprotein Maraba viral vector (a modified Maraba viral vector engineered to have the NotI restriction site downstream of the glycoprotein coding sequence) replacing the native Maraba glycoprotein.

In Vitro Neutralization with Whole Blood.

Rats were vaccinated with 1×10⁷ pfu of virus intravenously, two weeks prior to the terminal blood draw. On the day prior to the blood draw, half of the rats were depleted of complement with 35 U CVF. Blood was collected from rats using serum collection vacutainer tubes (BD Bioscience, San Jose, Calif.) and treated immediately with the anticoagulant Refludan (50 μg/mL). Blood was centrifuged at 800×g for 10 minutes to obtain plasma. Plasma aliquots were incubated for 30 minutes at 56° C. to inactivate complement. 200 μL of blood or fractions thereof were incubated for 1 hour at 37° C. with 2×10⁶ pfu of MRB LCMV G or MG1. Remaining infectious virus was quantified by plaque assay on Vero cells.

In Vitro Neutralization with Serum.

Female F344 Fischer rats were vaccinated with 1×10⁸ pfu of MRB LCMV G or MG1, with 1×10⁷ pfu of wild type (wt) Maraba, VSVd51, VSV LCMV G, MRB Lassa G or MRB Junin G intravenously. Serum was collected from rats 14 days post vaccination by cardiac puncture. Serum (25 μL) was heat inactivated (56° C. for 30 minutes) and used as a source of antibody. Complement was supplemented with an equal volume (25 μL) of rat serum (CompTech, Tyler, Tex.). Alternatively, 25 μL of dextrose gelatin veronal buffer (GVB⁺⁺; Lonza, Allendale, N.J.) was used. Serum was diluted into GVB⁺⁺ and neutralization was assessed following incubation with virus at a concentration of 5×10⁵ pfu per reaction for 1 hour at 37° C. Remaining infectious virus quantified by plaque assay on Vero cells. Neutralization was also assessed using rat serum (CompTech, Tyler, Tex.) pre-treated with 10 U/mL cobra venom factor (CVF; Quidel, San Diego, Calif.) for 1 hour at 37° C.

Cynomolgus macaques were treated with virus, either 1×10¹⁰ pfu intravenously (Animal 1) or 1×10⁹ pfu intracranially (Animal 2) under a protocol approved by the Animal Resource Centre, University Health Network, Toronto, ON, Canada. Serum was collected at various time points (pre, 8 days, 14 days, or 36 days postadministration). As described with rat and mouse immune serum, neutralization was assessed following incubation of heat inactivated immune serum (1 hour; 37° C.) with GVB⁺⁺ or with cynomolgus macaque serum (Innovative Research, Novi, Mich.). Data is expressed as the technical replicates ±standard deviation.

Neutralization of MRB LCMV G was assessed with rat immune serum supplemented with human serum (NHS) or serum immunodepleted of key complement components. C1q depleted, C3 depleted, and C5 depleted serum as well as NHS (ComTech, Tyler Tex.) or NHS pre-incubated (15 minutes at 37° C.) with the Compstatin analog, CP40 (25 μM) or Eculizumab (100 μg/mL) was combined with 25 μL of heat inactivated rat immune serum and 5×10⁵ pfu for 1 hour at 37° C. Immune rat serum that was combined with human C3 immuno-depleted serum originated from animals treated with CVF two days prior to blood draw.

In Vivo Animal Studies.

Female F344 Fischer rats weighing 100-150 g were purchased from Charles River (Wilmington, Mass.). All animals were housed in pathogen-free conditions and all studies conducted were in accordance with the guidelines of the Animal Care Veterinary Service facility of the University of Ottawa. Tumors were established by injecting 1×10⁶ 13762 MATBIII cells subcutaneously unilaterally or bilaterally in the left and right flanks. Animals were vaccinated with 1×10⁷ pfu of MG1 or MRB LCMV G intravenously, two weeks prior to their virus treatment. For the depletion of complement, 35 U of Cobra Venom Factor (CVF) (Quidel, San Diego, Calif.) was administered intraperitoneally, 24 hours prior to virus. To examine the stability of the virus early after administration, animals were treated intravenously with 1×10⁸ pfu of MG1 or MRB LCMV G and animals sacrificed 10 minutes post treatment. Blood was collected by cardiac puncture into EDTA vacutainer tubes (BD Bioscience, Mississauga ON) and tumors resected. Blood was titered on Vero cells to quantify remaining virus, and tumors were flash frozen, homogenized, and then titered on Vero cells to quantify infectious virus.

Virus naïve or vaccinated rats were also treated intratumorally with 1×10⁷ pfu of MG1 or MRB LCMV G. Tumors were collected 24 hours post virus treatment and immediately frozen. Infectious virus was quantified by plaque assay on Vero cells.

Results

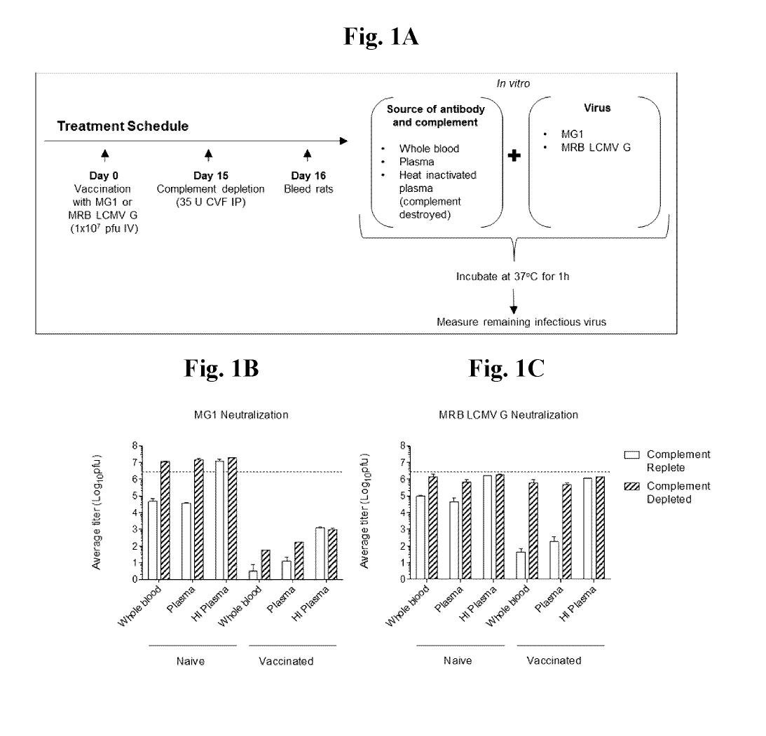

To investigate the effects of antibody and complement on MG1 (non-pseudotyped) and LCMV glycoprotein pseudotyped maraba viruses (MRB LCMV G), virus neutralization was assessed ex vivo in the blood of rats that were naïve to the viruses or that had been vaccinated two weeks prior to the blood draw (FIGS. 1A-C). The day prior to blood draw, half of the animals were depleted of complement using cobra venom factor (CVF). Blood was isolated from the animals, anti-coagulated with Refludan, and viral neutralization was assessed in vitro in whole blood, plasma, and heat inactivated plasma (complement destroyed). For blood collected from animals in the MG1 group, infectious MG1 was added in vitro to each of the blood fractions. For blood collected from animals in the MRB LCMV G group, infectious MRB LCMV G was added in vitro to each of the blood fractions. The blood-virus mixtures were incubated at 37° C. for 1 hour, after which point infectious virus remaining in the sample was assessed by virus plaque assay.

In the MG1 naïve blood and plasma, MG1 was sensitive to complement mediated neutralization. However when complement was destroyed by heat inactivation, neutralization of MG1 was not observed (FIG. 1B). In the blood collected from MG1 vaccinated animals, nearly 5-logs of virus was lost due to anti-MG1 antibody. The presence of complement in the whole blood, and plasma accounted for an additional reduction in virus titer as observed by comparing complement replete versus deplete, however this difference was very small. The same small difference was observed when comparing virus recovered in the heat-inactivated plasma, versus plasma samples. Thus, the antibody against native Maraba G was only modestly enhanced by complement. The data demonstrate that antibody generated against the native Maraba glycoprotein primarily neutralizes virus in a complement independent manner, as complement inhibition provided only modest increases in infectious virus titer.

MRB LCMV G was also moderately sensitive to complement neutralization in naïve blood (FIG. 1C). In contrast to MG1 however, blood collected from rats treated with the MRB LCMV G virus elicited antibodies that were only neutralizing if in the presence of complement. In the presence of complement (blood and plasma samples), nearly 4 logs of MRB LCMV G virus was neutralized. This effect was abrogated if the plasma was heat inactivated, or if the rats were pre-treated with complement inhibitor. The data demonstrates that antibodies generated against MRB LCMV G can only neutralize MRB LCMV G in the presence of complement.

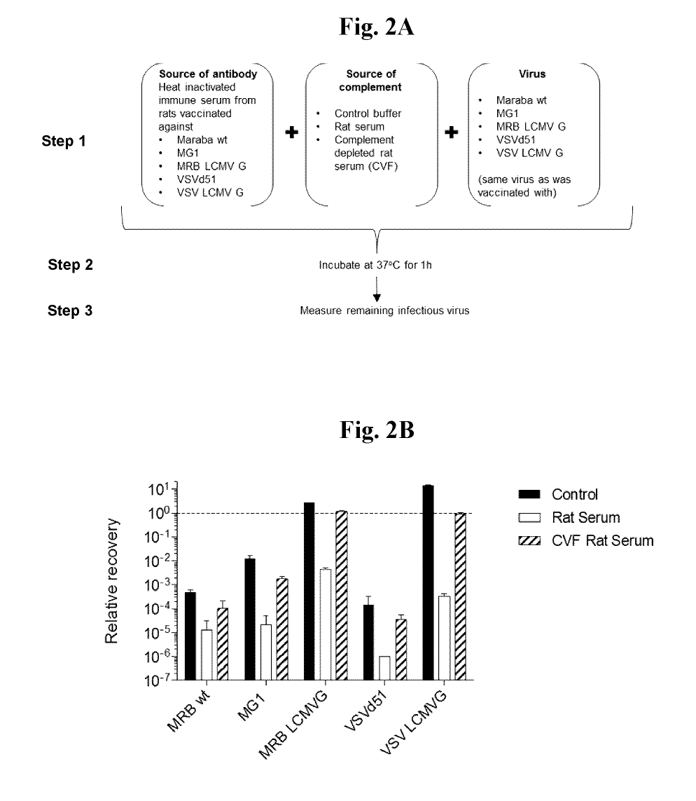

To establish that the complement-dependent phenotype of the antibody elicited by the MRB LCMV G virus was in fact independent of the rhabdovirus backbone, an ex-vivo rat study was performed. To generate a source of antibody, rats were vaccinated with wild type Maraba virus (Maraba wt), MG1, MRB LCMV G, as well as attenuated VSV (VSVd51), and VSV pseudotyped with the LCMV glycoprotein (VSV LCMV G). Two-weeks following vaccination, blood was collected from the rats and virus-specific antibody was prepared by heat-inactivating the collected serum. The serum (source of antibody) was combined with an active source of complement (naïve rat serum) or an inactive source of complement (naïve rat serum treated in vitro with CVF) or control buffer. To the antibody:complement mixture, the homologous virus was added, and neutralization was assessed by plaque assay following a one hour incubation at 37° C. See FIG. 2A.

For the viruses with native glycoproteins (Maraba wt, MG1, VSVd51), antibody collected from blood of vaccinated animals resulted in a significant reduction in virus titer, confirming that the native rhabdovirus glycoproteins elicited antibodies that were able to neutralize at least 99% of the input virus. The virus neutralization was enhanced only modestly in the presence of complement (rat serum), indicating that the majority of the antibody was neutralizing in a non-complement dependent manner. In contrast, for the LCMV G pseudotyped rhabdoviruses (MRB LCMV G and VSV LCMV G), antibody collected from the blood of vaccinated animals only resulted in a reduction in virus titer in the presence of complement (rat serum). These data are depicted at FIG. 2B and demonstrate that the antibodies targeting LCMV glycoprotein were non-neutralizing without complement but could mediate greater than 99% neutralization in the presence of complement. Moreover, the data indicate that this phenomenon is independent of the rhabdovirus backbone.

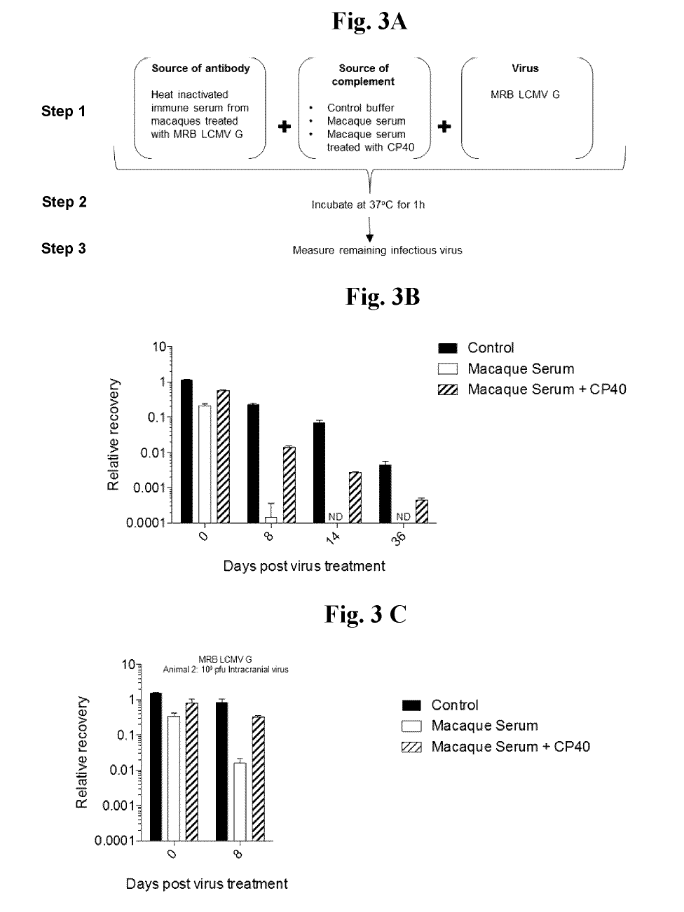

A cynomolgus macaque model was used to establish that the complement-dependent nature of the antibody neutralization was not a rodent specific phenomenon. Two animals were treated with MRB LCMV G, either intravenously, or intracranially. Their serum was collected at various time points after treatment. The serum was heat-inactivated to remove complement, and combined with two sources of complement: naïve macaque serum (active complement), naïve macaque serum treated with a complement inhibitor, CP40 (inactive complement), or control buffer. To each antibody:complement mixture, an equal amount of MRB LCMV G was added, and neutralization was assessed by plaque assay following a one hour incubation at 37° C. See FIG. 3A.

In both macaques, prior to vaccination, naïve monkey serum led to a small decrease in recoverable virus due to complement. As early as 8 days after vaccination with MRB LCMV G, the antibody-mediated, complement dependent neutralization was observed, leading to a loss of more than 99% of input virus. In both animals, complement inhibition with CP40 was able to greatly attenuate the effect of antibody. In one animal, this attenuation persisted over the course of 30 days following treatment. These data are depicted in FIGS. 3B and 3C and demonstrate that the complement-dependent nature of the LCMV G pseudotyped antibody neutralization is not a rodent-specific phenomenon.

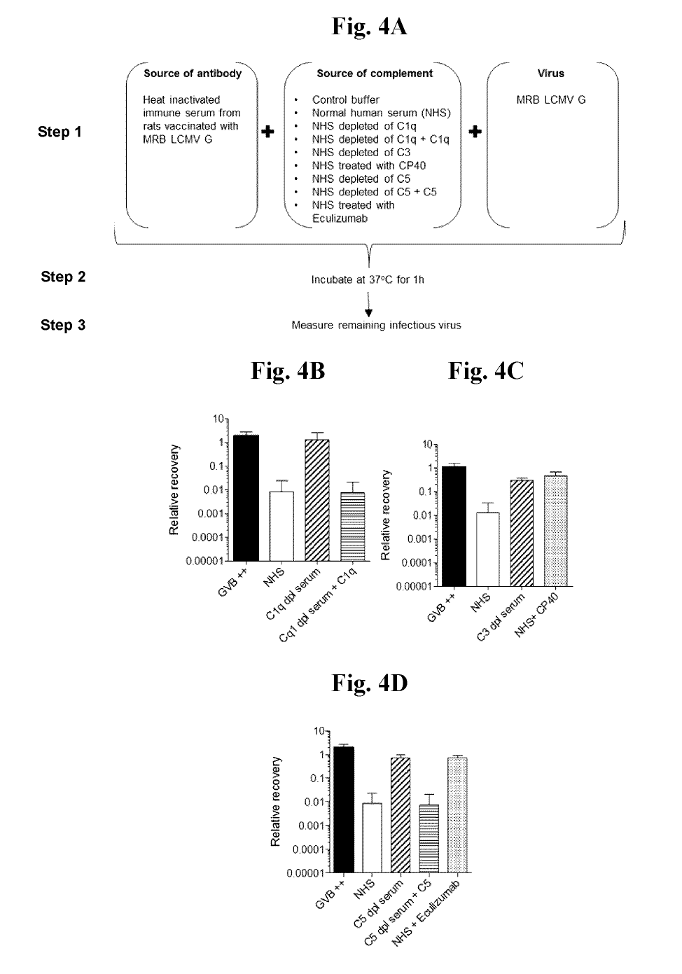

Using a human/rat ex vivo system, human complement inhibitors were evaluated to establish their efficacy in abrogating the complement-dependent MRB LCMV G virus neutralization. While the choice of reagents available to use in rats is limited to CVF, human complement inhibitors were evaluated in a partially human ex vivo system. Serum was collected from MRB LCMV G vaccinated rats. The serum was heat-inactivated to remove complement, and combined with control buffer (dextrose gelatin veronal buffer (GVB)), normal human serum (NHS) as a source of active complement, NHS treated with various complement inhibitors, or NHS depleted of key complement components (C1q, C3, or C5). For the latter mixture, additional add-back controls were included for C1q and C5, where these components were added to the depleted NHS to confirm that the observation was reversed. To the antibody:complement mixture, MRB LCMV G was added, and neutralization was assessed following a one hour incubation at 37° C. See FIG. 4A.

Antibody against the MRB LCMV G virus isolated from rat serum did not lead to neutralization of virus when combined with control buffer GVB. However, when combined with NHS (as a source of complement), the amount of recoverable virus was reduced to approximately 1% of input. If the key classical pathway molecule C1q was immunodepleted from NHS, the antibody and complement mediated neutralization of virus was abrogated. Upon addition of C1q to physiologic concentration, the neutralizing effect was restored. These data are depicted in FIG. 4B and demonstrate that virus neutralization was complement mediated via C1q (part of the Classical pathway). Similarly, when combined with NHS depleted of C3, the MRB LCMV G antibody isolated from rat serum did not result in virus neutralization. This effect was mirrored with addition of the C3 binding protein to the NHS (CP40). These data are depicted at FIG. 4C and demonstrate that virus neutralization was complement mediated via C3.

To assess whether the Terminal complement pathway is also involved in mediating the complement-dependent MRB LCMV G neutralization, C5 immunodepleted serum and the C5 inhibitory monoclonal antibody, Eculizumab were used. Both the depletion, and inhibition of C5 was able to prevent viral neutralization. However, this effect was reversed for C5 depleted serum, with the addition of physiological levels of C5. These data are depicted in FIG. 4D and demonstrate that virus neutralization was complement mediated via C5 (part of the Terminal pathway). Thus, the functional activity of the anti-LCMV G antibody can be abrogated by inhibiting of the classical complement pathway, the hub molecule C3 or the terminal complement pathway.

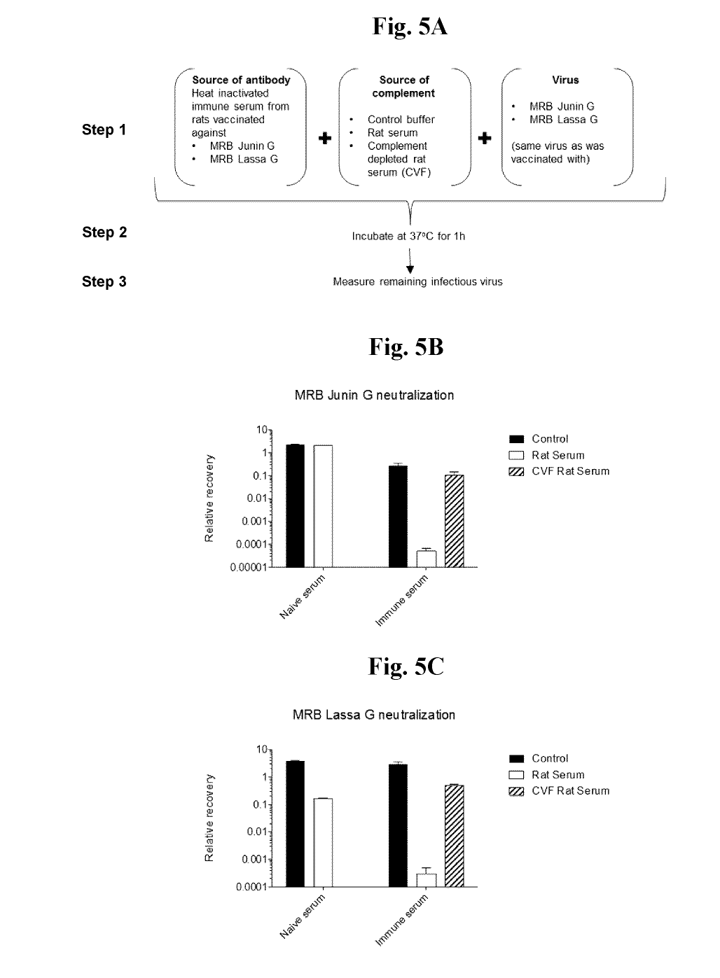

To test whether the complement-dependent neutralization observed for LCMV G-pseudotyped rhabdoviruses was specific to the LCMV glycoprotein or instead is a pan-arenavirus phenomenon, two new psuedotyped Maraba viruses were constructed: Maraba pseudotyped with Lassa virus glycoprotein (MRB Lassa G), and Maraba pseudotyped with Junin virus glycoprotein (MRB Junin G). The Lassa and Junin glycoproteins share 75 and 51 percent homology to the LCMV glycoprotein, respectively. Neutralization of the MRB Junin G and MRB Lassa G was evaluated ex vivo in the presence of heat inactivated immune serum from rats vaccinated against these viruses. The serum (source of antibody) was combined with one of two sources of complement: naïve rat serum (active complement), naïve rat serum treated in vitro with CVF (inactive complement), or control buffer. To the antibody:complement mixture, the homologous virus was added, and neutralization was assessed by plaque assay following a one hour incubation at 37° C. These data are depicted at FIG. 5A.

MRB Junin G incubated with heat inactivated naïve serum was not neutralized when combined with either control buffer (no complement) or rat serum (complement active). Antibody-containing immune serum collected from MRB Junin G vaccinated rats did not lead to neutralization in the control buffer; however when combined with naïve rat serum (active complement source) led to nearly 4 logs of virus being neutralized (relative recovery was 0.0001 of the input virus, corresponding to 0.01%). Neutralization was abrogated if the anti-MRB Junin G antibody was combined with serum depleted of complement with CVF. These data are depicted at FIG. 5B and demonstrate that the Junin glycoprotein elicits antibodies that require complement activity to neutralize virus.

In the MRB Lassa G naïve serum, there was a small decrease in virus recovered from samples containing rat serum (complement active) versus control buffer (no complement). Serum collected from MRB Lassa G vaccinated rats (source of antibodies) did not lead to neutralization in the control buffer, however when combined with naïve rat serum (active complement source) over 3 logs of virus was neutralized (relative recovery was 0.001 of the input virus, corresponding to 0.1%). This effect was abrogated if the rat serum was pre-treated with the complement inhibitor, CVF. These data are depicted at FIG. 5C and demonstrate that the Lassa glycoprotein also elicits antibodies that require complement activity to neutralize virus.

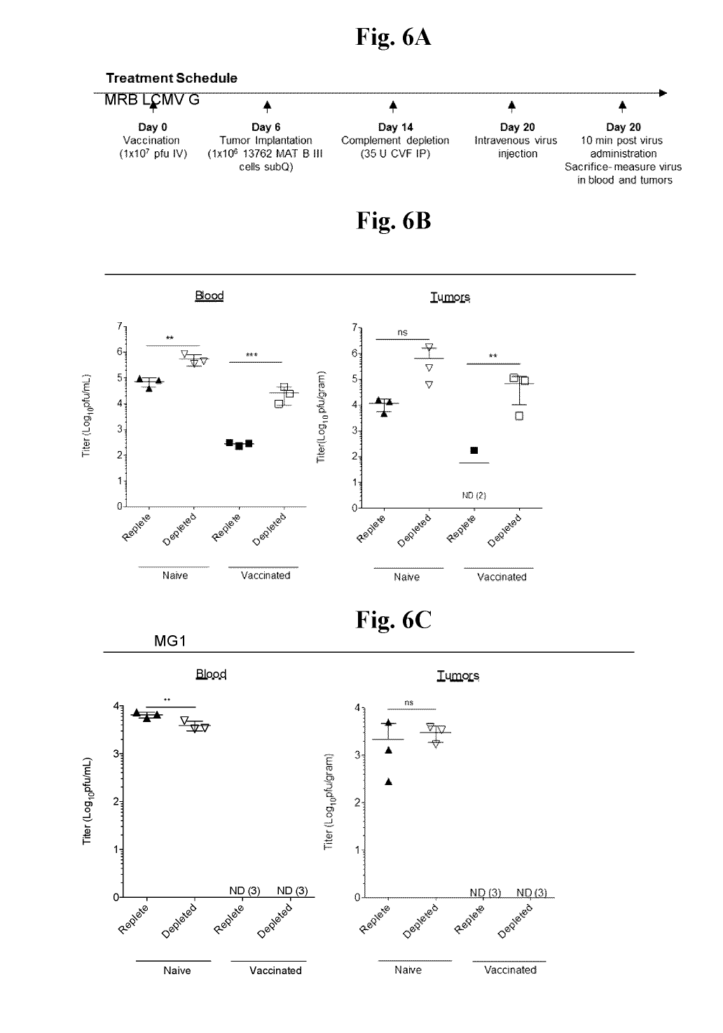

Cobra Venom Factor (CVF) acts as a C3b mimetic and combines to produce a C3 convertase that activates and depletes the C3 molecule. This depletion is analogous to targeting the C3 molecule with compounds such as CP40. Using a Fischer rat model to which the mammary adenocarcinoma cell line 13762 MAT B III is syngeneic, the ability of complement depletion to increase the stability of MG1 and MRB LCMV G viruses in the blood as well as increase delivery to tumors was evaluated. Briefly, virus vaccinated or naïve rats were implanted with bilateral mammary adenocarcinoma tumours (13762 MAT B III). CVF was used to deplete complement in a subset of the animals, and virus was subsequently delivered intravenously (tail vein injection). Animals were sacrificed 10 minutes after virus administration to quantify virus in the blood, and tumours by plaque assay. See FIG. 6A.

In the MRB LCMV G naïve rats, there was a significant increase in infectious virus recovered from the blood of complement-depleted rats compared to complement-replete rats. Significantly less infectious virus was recovered from the blood of vaccinated animals relative to naïve animals. In contrast, a significant increase (average 97-fold increase) in infectious virus recovery from the blood of MRB LCMV G immune animals was observed if they were treated with CVF complement inhibitor prior to intravenous MRB LCMV G. These findings translated to a corresponding significant increase in infectious virus recovered from subcutaneous tumours in the MRB LCMV G immune animals, and a trend toward increased recovery in tumours of naive animals that was associated with complement depletion. These data are depicted at FIG. 6B and demonstrate that complement inhibition in vivo leads to enhanced stability of MRB LCMV G virus in the blood, which is correlated with increased virus recovered from tumours.

In contrast to Maraba virus pseudotyped with the LCMV glycoprotein, no benefit from complement depletion on stability in the blood or delivery to tumors was observed for MG1 in either naïve or immune animals. For the MG1 rats, when comparing virus recovered from blood isolated from MG1 naïve rats versus MG1 vaccinated rats, there was a dramatic decrease in the amount of virus recovered. In the blood isolated from MG1 vaccinated rats, there was no recoverable virus in the blood—and this antibody neutralization was not found to be complement mediated, since treatment with CVF (complement depletion) did not abrogate the effect. Similar observations were observed in the tumours. These data are depicted at FIG. 6C and demonstrate that the complement-dependent antibody neutralization observed with MRB LCMV G is glycoprotein specific.

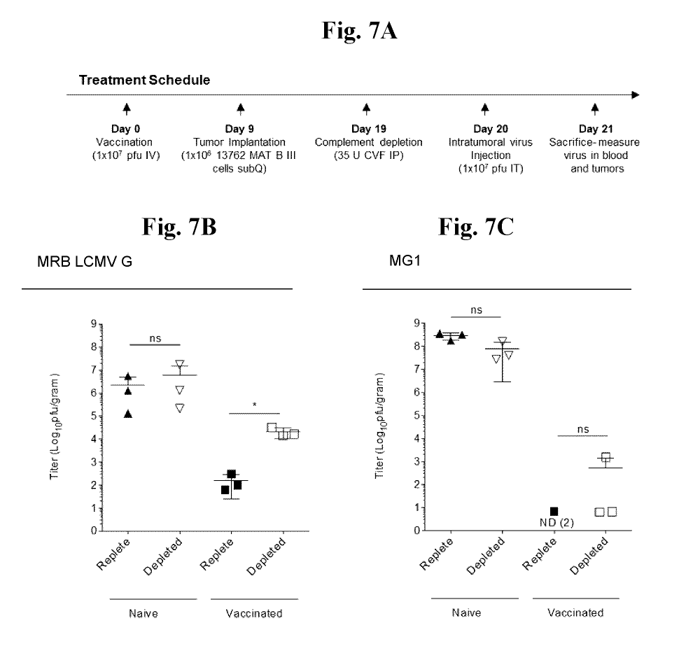

The effect of complement depletion was also assessed in the context of a local administration of virus. Naïve and vaccinated rats were treated with CVF or sham and subsequently given an intratumoral dose of MG1 or MRB LCMV G virus according to the schedule in FIG. 7. Complement depletion increased the titer of MRB LCMV G that was recovered from tumors from immune rats 24 hours after virus administration (mean 135-fold increase), but not naïve rats following an intratumoral injection of virus. Consistent with the study on viral stability in the blood, the antibodies against MG1 neutralized the virus independently of complement to prevent infection of tumors. Complement depletion also did not aid in the infection of MG1 of subcutaneous tumors in naïve animals. Thus, complement plays an important role both in the blood stream and in the tumor microenvironment to limit infection of rhabdoviruses pseudotyped with arenavirus glycoproteins. A combination complement inhibition and pseudotyping strategy enables the local and systemic delivery of infectious virus to tumors, despite the presence of antiviral antibody.

### DISCUSSION

Within the first week following administration of rhabodviruses such as VSV and Maraba virus, neutralizing antibodies are generated against these viruses, limiting multiple rounds of dosing. In contrast, arenaviruses such as LCMV are known for their inability to generate early neutralizing antibodies. This property has been consferred to rhabdoviruses by pseudotyping and when tested in mice, a VSV virus pesudotyped with an LCMV glycoprotein did not elicit a strong neutralizing antibody response and demonstrated enhanced delivery to tumours following multiple therapeutic doses. However, this strategy has not translated to other animal models. Using rhabdoviruses pseudotyped with arenavirus glycoproteins in rat and primate models, the present application surprisingly demonstrates for the first time that early antibodies are generated against the arenavirus glycoproteins in three different species which, while non-neutralizing on their own, mediate robust complement-dependent viral neutralization, limiting the therapeutic potential of these viruses. Specifically, antibody binding to virus pseudotyped with arenavirus glycoproteins mediates C1q binding and neutralization via the membrane attack complex. The present application demonstrates that complement inhibition improves the stability and delivery of such pseudotyped rhabdoviruses to tumors whether administered locally by intratumoral injection or systemically by intravenous injection, leading to a persistent increase in the oncolytic infection of tumours in both naïve and immune animals. The present application supports the use of a complement inhibitor to evade virus neutralization in immune animals or humans, leading to an increased therapeutic effect when administered as a single dose and enabling multiple rounds of therapeutic pseudotypedviruses to be effectively administered.

1-42. (canceled)

43. A replicative oncolytic pseudotype rhabdovirus comprising an arenavirus glycoprotein.

44. The replicative oncolytic rhabdovirus of claim 43, wherein the pseudotyped replicative oncolytic rhabdovirus has a wild type or genetically modified Vesiculovirus backbone.

45. The replicative oncoytic rhabdovirus of claim 44, wherein the pseudotyped replicative oncolytic rhabdovirus has a wild type or genetically modified VSV or Maraba virus backbone.

46. The replicative oncolytic rhabdovirus of claim 44, wherein the pseudotyped replicative oncolytic rhabdovirus has a wild type or genetically modified Maraba virus backbone.

47. The replicative oncolytic rhabdovirus of claim 44, wherein the pseudotyped replicative oncolytic rhabdovirus has a wild type or genetically modified VSV virus backbone.

48. The replicative oncolytic rhabdovirus of claim 43, wherein the pseudotyped oncolytic rhabdovirus expresses a tumor antigen.

49. The replicative oncolytic rhabdovirus of claim 43, wherein the tumor antigen is a tumor associated antigen selected from the group consisting of MAGEA3, Human Papilloma Virus E6/E7 fusion protein, human Six-Transmembrane Epithelial Antigen of the Prostate protein, Cancer Testis Antigen 1, and a variant thereof.

50. The replicative oncolytic rhabdovirus of claim 43, wherein the replicative oncolytic rhabdovirus is pseudotyped with an Old World arenavirus glycoprotein.

51. The replicative oncolytic rhabdovirus of claim 50, wherein the Old World arenavirus glycoprotein is selected from the group consisting of an LCMV, Lassa virus, Mopeia virus, Mobala virus, Ippy virus, Mariental virus, Merino Walk virus, Menekre virus, Gairo virus, Gbagroube virus, Morogoro virus, Kodoko virus, Lunk virus, Okahandja virus, Lujo virus, Lemniscomys virus, Mus minutoides virus, Wenzhou virus, and Luna virus glycoprotein.

52. The replicative oncolytic rhabdovirus of claim 50, wherein the Old World arenavirus glycoprotein is a Lassa virus glycoprotein.

53. The replicative oncolytic rhabdovirus of claim 50, wherein the Old World arenavirus glycoprotein is not an LCMV virus glycoprotein.

54. The replicative oncolytic rhabdovirus of claim 43, wherein the replicative oncolytic rhabdovirus is pseudotyped with a New World arenavirus glycoprotein.

55. The replicative oncolytic rhabdovirus of claim 54, wherein the New World arenavirus glycoprotein is selected from a Junin virus, Tacaribe virus, Machupo virus, Cupixi virus, Amapari virus, Parana virus, Patawa virus, Tamiami virus, Pichinde virus, Latino virus, Flexal virus, Guanarito virus, Sabia virus, Oliveros virus, Whitewater Arroyo virus, Pirital virus, Pampa virus, Bear Cany one virus, Ocozocoautla de Espinosa virus, Allpahuayo virus, Tonto Creek virus, Big Brushy Tank virus, Real de Catorce virus, Catarina virus, Skinner Tank virus, and Chapare virus glycoprotein.

56. The replicative oncolytic rhabdovirus of claim 43, wherein the New World arenavirus glycoprotein is a Junin virus glycoprotein.

57. A replicative oncolytic virus comprising M, P, N and L proteins from an attenuated VSV or Maraba virus, and an arenavirus glycoprotein.

58. A pharmaceutical composition comprising an effective amount of a pseudotyped replicative oncolytic rhabdovirus according to claim 43 and a pharmaceutically acceptable adjuvant, diluent or carrier.

59. A method for treating and/or preventing cancer or a metastasis in a mammal in need thereof, comprising administering to the mammal an effective amount of a combination comprising (a) a replicative oncolytic rhabdovirus pseudotyped with an arenavirus glycoprotein and (b) one or more complement inhibitors.

60. The method of claim 59, wherein the pseudotyped replicative oncolytic rhabdovirus has a wild type or genetically modified Vesiculovirus backbone.

61. The method of claim 60, wherein the pseudotyped replicative oncolytic rhabdovirus has a wild type or genetically modified VSV or Maraba virus backbone.

62. The method of claim 61, wherein the pseudotyped replicative oncolytic rhabdovirus has a wild type or genetically modified Maraba virus backbone.

63. The method of claim 60, wherein the pseudotyped replicative oncolytic rhabdovirus has a wild type or genetically modified VSV virus backbone.

64. The method of claim 59, wherein the complement inhibitor is an inhibitor of the classical complement pathway.

65. The method of claim 64, wherein the complement inhibitor targets C1, optionally selected from a C1 esterase inhibitor (Cinryze or Berinert), and an anti-C1s antibody such as TNT009 or TNT010.

66. The method of claim 59, wherein the complement inhibitor is an inhibitor of the alternative complement pathway.

67. The method of claim 66, wherein the complement inhibitor targets complement factor B (CFB) and/or complement factor D (CFD), optionally selected from an antibody or antibody fragment such as TA106, FCFD4514S, and lampalizumab, an anti-CFB siRNA, an anti-CFD siRNA, and an aptamer.

68. The method of claim 59, wherein the complement inhibitor is an inhibitor of both the classical and alternative complement pathways.

69. The method of claim 68, wherein the complement inhibitor targets C3, optionally selected from TT30 (CR2/CFH), MiniCFH, sCR1 (CDX-1135), Microcept (APT070), TT32 (CR2/CR1), an antibody such as HI 7, compstatin or an analog, peptidomimetic, or derivative thereof such as 4(1MeW)/POT-4, 4(1MeW)/APL-1/2, Cp40/AMY-101, and PEG-Cp40.

70. The method of claim 69, wherein the complement inhibitor targets C5, optionally selected from an antibody or antigen binding fragment thereof such as Eculizumab, LFG316, Mubodina, CaCP29 and Pexelizumab, recombinant proteins such as Coversin (OMCl), an aptamer such as ARC1005, and ARC1905, an anti-C5 siRNA such as ALN-CC5 and a C5a receptor antagonist such as NGD 2000-1, CCX168, PMX53 and AcPhe[Orn-Pro-D-Cyclohexylalanine-Trp-Arg] (AcF-[OpdChaWR].

71. The method of claim 59, wherein the pseudotyped replicative oncolytic rhabdovirus is administered to the mammal in combination with at least two complement inhibitors.

72. The method of claim 71, wherein the pseudotyped replicative oncolytic rhabdovirus is administered in combination with an inhibitor of the classical complement pathway and either an inhibitor of the alternative complement pathway or an inhibitor of the terminal pathway.

73. The method of claim 59, wherein the pseudotyped replicative oncolytic rhabdovirus and the complement inhibitor are administered simultaneously.

74. The method of claim 59, wherein the pseudotyped replicative oncolytic rhabdovirus and the complement inhibitor are administered sequentially and wherein a first administration of pseudotyped oncolytic rhabdovirus occurs prior to a first administration of complement inhibitor.

75. The method of claim 74, wherein the first administration of pseudotyped oncolytic rhabdovirus occurs within 30 days of a first administration of complement inhibitor.

76. The method of claim 59, wherein the pseudotyped replicative oncolytic rhabdovirus is administered multiple times over a period of at least 8 days and wherein a first administration of complement inhibitor occurs prior to a second or subsequent administration of pseudotyped replicative oncolytic rhabdovirus.

77. The method of claim 59, wherein the pseudotyped replicative oncolytic rhabdovirus and the complement inhibitor are administered sequentially and wherein a first administration of complement inhibitor occurs prior to a first administration of pseudotyped replicative oncolytic virus and preferably occurs within 30 days of a first administration of pseudotyped replicative oncolytic virus.

78. The method of claim 59, wherein the pseudotyped replicative oncolytic rhabdovirus is administered multiple times.

79. The method of claim 43, wherein the pseudotyped oncolytic rhabdovirus expresses a tumor antigen.

80. The method of claim 79, wherein the tumor antigen is a tumor associated antigen selected from the group consisting of MAGEA3, Human Papilloma Virus E6/E7 fusion protein, human Six-Transmembrane Epithelial Antigen of the Prostate protein, Cancer Testis Antigen 1, and a variant thereof.

81. The method of claim 79, wherein the mammal has a pre-existing immunity to the tumor associated antigen.

82. The method of claim 81, wherein the pre-existing immunity in the mammal is established by administering said tumor associated antigen to the mammal prior to administering the pseudoytped replicative oncolytic rhabodvirus.

83. The method of claim 59, wherein the replicative oncolytic rhabdovirus is pseudotyped with an Old World arenavirus glycoprotein.

84. The method of claim 83, wherein the Old World arenavirus glycoprotein is selected from the group consisting of an LCMV, Lassa virus, Mopeia virus, Mobala virus, Ippy virus, Mariental virus, Merino Walk virus, Menekre virus, Gairo virus, Gbagroube virus, Morogoro virus, Kodoko virus, Lunk virus, Okahandja virus, Lujo virus, Lemniscomys virus, Mus minutoides virus, Wenzhou virus, and Luna virus glycoprotein.

85. The method of claim 83, wherein the Old World arenavirus glycoprotein is an LCMV or Lassa virus glycoprotein.

86. The method of claim 59, wherein the replicative oncolytic rhabdovirus is pseudotyped with a New World arenavirus glycoprotein.

87. The method of claim 86, wherein the New World arenavirus glycoprotein is selected from a Junin virus, Tacaribe virus, Machupo virus, Cupixi virus, Amapari virus, Parana virus, Patawa virus, Tamiami virus, Pichinde virus, Latino virus, Flexal virus, Guanarito virus, Sabia virus, Oliveros virus, Whitewater Arroyo virus, Pirital virus, Pampa virus, Bear Cany one virus, Ocozocoautla de Espinosa virus, Allpahuayo virus, Tonto Creek virus, Big Brushy Tank virus, Real de Catorce virus, Catarina virus, Skinner Tank virus, and Chapare virus glycoprotein.

88. The method of claim 86, wherein the New World arenavirus glycoprotein is a Junin virus glycoprotein.

89. The method of claim 59, wherein the pseudotyped replicative oncolytic rhabdovirus is administered as one or more doses of 10⁶-10¹⁴ pfu, 10⁶-10¹² pfu, 10⁸-10¹⁴ pfu or 10⁸-10¹² pfu.

90. The method of claim 89, wherein the pseudotyped replicative oncolytic rhabdovirus is administered at least twice at a dose of 10⁶-10¹⁴ pfu, 10⁶-10¹² pfu, 10⁸-10¹⁴ pfu or 10⁸-10¹² pfu over a period of at least 8 days.

91. The method of claim 59, wherein the pseudotyped replicative oncolytic rhabdovirus is administered intravascularly and/or intratumoraly.

92. The method of claim 59, wherein the cancer is colorectal cancer, lung cancer, esophageal cancer, melanoma, pancreatic cancer, ovarian cancer, renal cell carcinoma, cervical cancer, liver cancer, breast cancer, head and neck cancer, prostate cancer, brain cancer, bladder cancer and soft tissue sarcoma.

93. The method of claim 59, wherein the complement inhibitor is an antibody or antibody fragment and is administered as one or more doses of 0.01-10 mg/kg, 0.1-10 mg/kg, 1-10 mg/kg, 2-8 mg/kg, 3-7 mg/kg, 4-5 mg/kg or at least 10 mg/kg.

94. The method of claim 93, wherein the complement inhibitor is administered at least three times per week, at least four times per week, at least five times per week, weekly, biweekly, every other week, or every three weeks.

95. The method of claim 94, wherein the mammal is a human.

96. The method of claim 95, wherein the human has a cancer that is refractory to one or more previous treatment regimens.


SEQUENCE LISTING (4 sequences)

SEQ ID NO: 1 - DNA, 1492 bp, Artificial Sequence
  Codon-optimized Junin glycoprotein coding sequence
ggtacccagttatatttgttacaacaatgggacaattcatctccttcatgcaggagatacctactttcctccaagaggctctcaatatcgctctggtggcggtttcactgatcgctatcataaagggcattgtgaacttgtacaaatcaggcctgttccaattctttgtgttcctggctcttgcagggagatcttgtacagaagaggcttttaaaatcggcctccacactgagtttcagaccgtgagtttctcaatggtcggcctgttttcaaataatccccatgacctgcccctgttgtgtaccctgaacaagagtcacctgtacatcaagggcggaaacgcatcattcatgatctcctttgacgatattgaagtgctgctgcctcaatacgatgtgataatacagcacccagccgacatgtcctggtgcagcaagtccgatgaccaaatttggttgtcccagtggtttatgaatgcagtcggacatgattggcacttggacccacccttcctttgccgcaatagaactaagaccgagggtttcatttttcaggtcaacacaagcaagactggggtcaacgaaaactatgcaaaaaagttcaagacaggtatgcatcacctctaccgggagtaccctgattcttgcctgaacgggaagttgtgcctgatgaaggcccagccaacgtcctggcctctgcagtgccctttggaccatgtgaacactttgcactttctcactagaggcaaaaacatccagctccctaggcgatcccttaaggcgttcttttcttggagtctgacggattcttccggaaaggacacccctgggggctactgtctcgaagaatggatgctggtagctgcaaagatgaaatgttttgggaacactgccgtcgcgaaatgcaacctgaaccatgattctgaattttgcgatatgctccgacttttcgactataataagaatgctatcaagacactgaacgatgaaactaagaaacaggtgaatctcatgggacagaccattaatgctctgatcagtgacaatctgctgatgaagaataaaatccgagagctgatgtcagtgccctattgtaattatacaaaattttggtacgtgaatcacacactgtccggccagcactctctgccgaggtgctggctgattaagaataatagctacttgaacatcagcgacttcagaaacgactggattctcgagtccgattttctgatcagcgaaatgctcagtaaagagtattcagacagacagggcaagacaccccttactctcgttgatatttgtttttggagtacagttttttttacggcctccctgttcctccatctggtcggtattcctacccaccgacatatccgcggcgaggcatgtccactgcctcatcgcctcaattcactgggaggctgtcgatgtggaaagtatccgaatctcaaaaaacctaccgtctggcgcagaagacattaggcggccgc

SEQ ID NO: 2 - protein, 485 aa, Mammarenavirus juninvirus
MGQFISFMQEIPTFLQEALNIALVAVSLIAIIKGIVNLYKSGLFQFFVFLALAGRSCTEEAFKIGLHTEFQTVSFSMVGLFSNNPHDLPLLCTLNKSHLYIKGGNASFMISFDDIEVLLPQYDVIIQHPADMSWCSKSDDQIWLSQWFMNAVGHDWHLDPPFLCRNRTKTEGFIFQVNTSKTGVNENYAKKFKTGMHHLYREYPDSCLNGKLCLMKAQPTSWPLQCPLDHVNTLHFLTRGKNIQLPRRSLKAFFSWSLTDSSGKDTPGGYCLEEWMLVAAKMKCFGNTAVAKCNLNHDSEFCDMLRLFDYNKNAIKTLNDETKKQVNLMGQTINALISDNLLMKNKIRELMSVPYCNYTKFWYVNHTLSGQHSLPRCWLIKNNSYLNISDFRNDWILESDFLISEMLSKEYSDRQGKTPLTLVDICFWSTVFFTASLFLHLVGIPTHRHIRGEACPLPHRLNSLGGCRCGKYPNLKKPTVWRRRH

SEQ ID NO: 3 - DNA, 1507 bp, Artificial Sequence
  Codon-optimized Lassa glycoprotein coding sequence
ggtacccagttatatttgttacaacaatgggacaaatcatcacgtttttccaggaagtgccccacgtcatagaggaggtaatgaatatagtgctcattgccctcagtttgctggcgatcctgaaagggatctacaacgtggcgacttgtggtctgtttggcttggtgtctttcctgctgttgtgcggtcgaagctgcagtaccacctataagggagtctacgagctgcagacactggaactggacatggctagcttgaacatgactatgcctctctcctgcacaaagaataacagtcaccattacataatggtggggaatgaaactggtttggaactcacacttaccaacacatccatcataaatcacaagttttgtaacctcagtgacgcccacaaaaaaaacttgtatgatcacgctctcatgtccataatcagcacttttcacctgtctatccctaacttcaatcagtacgaggctatgtcttgcgactttaacgggggcaaaatcagcgtgcaatacaatctgagccacgcatatgccgtcgacgccgccaaccactgcggaactatcgctaacggcgtcctgcagacattcatgcggatggcttggggcggctcctatatcgctctggatagcggaaagggcagttgggactgtattatgacctcataccagtaccttattatccagaacaccacctgggaggatcactgtcaattttcccggccgtccccaatcggctatctgggcctcctgagccaaagaactcgggacatttacatatctcggcgactcctcgggacattcacatggaccctgtccgactctgaagggaatgaaacgccaggcgggtattgcctgacccgatggatgctgatcgaagccgagctcaagtgctttggaaataccgcagtcgccaagtgtaatgaaaagcatgatgaagaattttgcgatatgctgcggctgttcgatttcaataaacaggccattcgacggctgaaaaccgaggcccaaatgagtatccagctgattaacaaggccgttaatgccctgattaatgaccagctcattatgaaaaatcacctgcgggatatcatgggcattccttactgtaactattccaagtattggtatctgaaccacaccgtgactggcaaaacgtcactgccaaggtgctggctggtctccaatggaagctacctgaacgagacccatttttccgatgatatcgagcagcaggccgataatatgattaccgaactgttgcagaaagaatacatggaccgccagggcaaaactccacttgggttggtcgacctgtttgtgttctctaccagcttctacttgattagcattttcctgcacctggtgcgcatccccacgcacagacatgtcatcggtaagccatgccctaagccgcatagactcaaccatatggggatttgctcctgtggtctctataaacaccccggcgtgcctgtcaaatggaagaggtgagcggccgc

SEQ ID NO: 4 - protein, 490 aa, Mammarenavirus lassavirus
MGQIITFFQEVPHVIEEVMNIVLIALSLLAILKGIYNVATCGLFGLVSFLLLCGRSCSTTYKGVYELQTLELDMASLNMTMPLSCTKNNSHHYIMVGNETGLELTLTNTSIINHKFCNLSDAHKKNLYDHALMSIISTFHLSIPNFNQYEAMSCDFNGGKISVQYNLSHAYAVDAANHCGTIANGVLQTFMRMAWGGSYIALDSGKGSWDCIMTSYQYLIIQNTTWEDHCQFSRPSPIGYLGLLSQRTRDIYISRRLLGTFTWTLSDSEGNETPGGYCLTRWMLIEAELKCFGNTAVAKCNEKHDEEFCDMLRLFDFNKQAIRRLKTEAQMSIQLINKAVNALINDQLIMKNHLRDIMGIPYCNYSKYWYLNHTVTGKTSLPRCWLVSNGSYLNETHFSDDIEQQADNMITELLQKEYMDRQGKTPLGLVDLFVFSTSFYLISIFLHLVRIPTHRHVIGKPCPKPHRLNHMGICSCGLYKHPGVPVKWKR